(19) United States
(12) Patent Application Publication
(10) Pub. No.: US 2024/0011023 A1
(43) Pub. Date: Jan. 11, 2024

(54) HYBRIDIZED GUIDE NUCLEIC ACIDS FOR USE WITH TEMPLATE-BASED GENE EDITORS

(71) Applicant: Verve Therapeutics, Inc., (US)

(72) Inventors: Vidya Subramanian, Cambridge, MA (US); Kallanthottathil G. Rajeev, Wayland, MA (US); Hariharan Jayaram, Cambridge, MA (US); Caroline Reiss, Concord, MA (US); Maryam Habibian, Newton, MA (US); Gitali Devi, North Grafton, MA (US); Allison Baker, Brookline, MA (US)

(21) Appl. No.: 18/219,643

(22) Filed: Jul. 7, 2023

Related U.S. Application Data
(60) Provisional application No. 63/359,558, filed on Jul. 8, 2022.

(51) Int. Cl. C12N 15/11; C12N 9/22

(52) U.S. Cl. CPC C12N 15/11; C12N 9/22; C12N 2310/20

(57) ABSTRACT

Polynucleotides are engineered to hybridize to one another to form a functional guide nucleic acid having an editing template. The hybridized guide nucleic acids may be used with template-based gene editors to write an edit into a target genomic location using the editing template as a template for the edit.

### RELATED APPLICATION

This application claims the benefit of U.S. Provisional Application No. 63/359,558 filed on Jul. 8, 2022, which is hereby incorporated herein in its entirety to the extent that it does not conflict with the disclosure presented herein.

### INTRODUCTION

Gene editor systems have the potential to transform modern medicine by offering in vivo and ex vivo treatments and cures for genetic diseases. Gene editor systems typically include a guide RNA (“gRNA”) molecule, which functions to guide a gene editor protein or complex (“gene editor”) to a desired genome location to facilitate the intended editing. Naturally occurring gRNA includes two polynucleotides: a spacer sequence which guides the gene editor to a desired location in a gene, and a tracr region sequence which is bound by the gene editor. Synthetic gene editor systems frequently use “single guide” RNA (sgRNA) designs that combine the spacer region and tracr region sequences into a single polynucleotide, which sgRNAs may have one or more advantages relative to gRNAs formed from two different RNA molecules. sgRNAs may be modified to enhance activity without fear that the modifications will adversely affect hybridization of the separate spacer region and tracr region molecules. In addition, synthesis of a sgRNA is less complex than synthesizing and hybridizing two separate RNA molecules. Alternate designs for gRNA assembly including editing templates for use with template-based editors are described herein.

### SUMMARY

Described herein, among other things, are hybridized guide nucleic acids for use with template-based gene editors and methods for making and using the same. The guide nucleic acids comprise two or more polynucleotides that hybridize to one another to form a functional guide nucleic acid.

Production of guide nucleic acids by hybridizing multiple polynucleotides may be advantageous for assembly of guide nucleic acid libraries to test many sequence options in parallel. Additionally, production of shorter guide nucleic acid molecules may improve manufacturing quality. Methods for modular assembly of guide nucleic acids molecules from multiple polynucleotides are desired.

In various aspects, the present disclosure describes a hybridized guide nucleic acid for use with a template-based gene editor. The hybridized guide nucleic acid comprises a first polynucleotide comprising a first guide nucleic acid element and a first hybridization sequence; and a second polynucleotide comprising a second guide nucleic acid element and a second hybridization sequence. The second guide nucleic acid element is an editing template. The first and second hybridization sequences are complementary. The first and second hybridization sequences hybridizes to one another to form the hybridized guide nucleic acid.

In various aspects, the present disclosure describes a hybridized guide nucleic acid for use with a template-based gene editor. The hybridized guide nucleic acid comprises a plurality of polynucleotides. Each polynucleotide comprises at most 80 nucleotides in length. Each polynucleotide comprises a guide nucleic acid element and a hybridization sequence. Each polynucleotide comprises a guide nucleic acid element and a hybridization sequence. At least one of the polynucleotides comprises an editing template. Each hybridization sequence is complementary to at least one other hybridization sequence. Each of the plurality of polynucleotides hybridizes to another of the plurality of polynucleotides to form the hybridized guide nucleic acid.

### BRIEF DESCRIPTION OF THE FIGURES

FIG. 1A is a schematic drawing illustrating a template-based gene editing process using a single guide RNA.

FIG. 1B is a schematic drawing illustrating a template-based gene editing process using a hybridized guide RNA.

FIG. 1C is a schematic drawing illustrating a template-based gene editing process using a split guide and split editor.

FIG. 1D is a schematic drawing illustrating the use of a nicking guide RNA (ngRNA) after the initial template-based editing, as illustrated in, for example, FIG. 1A, FIG. 1B, or FIG. 1C has occurred.

FIG. 2 is a schematic drawing of an embodiment of a single guide RNA (sgRNA).

FIG. 3 is a schematic drawing of an embodiment of a template-based gene editing guide RNA (pegRNA).

FIG. 4 is a schematic drawing of an embodiment of a modular hybridized guide nucleic acid.

FIG. 5 is a schematic drawing of an embodiment of a modular hybridized guide nucleic acid.

FIG. 6 is a schematic drawing of an embodiment of a modular hybridized guide nucleic acid.

FIG. 7 is a schematic drawing of an embodiment of a modular hybridized guide nucleic acid.

FIG. 8 is a schematic drawing of an embodiment of a modular hybridized guide nucleic acid.

FIG. 9 is a schematic drawing of an embodiment of a modular hybridized guide nucleic acid.

FIG. 10 shows the results of screening rounds 1 and 2. (A) Screening Round 1 of different hybridized guide sequences. Data from Huh7 Cells at 315 ng/mL total RNA dose using mRNA Configuration 1 (2 mRNAs, one of which includes and MCP-RT fusion protein). (B) Screening Round 1 of different hybridized guide sequences. Data from Huh7 Cells at 1260 ng/mL total RNA dose using mRNA Configuration 1 (2 mRNAs, one of which includes and MCP-RT fusion protein). (C) Screening Round 1 of different hybridized guide sequences. Data from Huh7 Cells at 315 ng/mL total RNA dose using mRNA Configuration 2 (1 mRNA encoding PEmax). (D) Screening Round 1 of different hybridized guide sequences. Data from Huh7 Cells at 1260 ng/mL total RNA dose using Configuration 2 (1 mRNA encoding PEmax). (E) Screening Round 2 of different hybridized guide sequences. Data from Huh7 Cells at 157.5 ng/mL total RNA dose using mRNA Configuration 1 (2 mRNAs, one of which includes and MCP-RT fusion protein). (F) Screening Round 2 of different hybridized guide sequences. Data from Huh7 Cells at 315 ng/mL total RNA dose using mRNA Configuration 1 (2 mRNAs, one of which includes and MCP-RT fusion protein).

### DETAILED DESCRIPTION OF ILLUSTRATIVE EMBODIMENTS

Described herein, among other things, are hybridized guide nucleic acids for use with template-based gene editors and methods for making the same. The guide nucleic acids comprise two or more polynucleotides that hybridize to one another to form a functional guide nucleic acid. In some embodiments, the hybridized guide nucleic acid includes an editing template. In some embodiments, the hybridized guide nucleic acid or one or more of the polynucleotides forming the hybridized guide nucleic acids is chemically modified. In some embodiments, the hybridized guide nucleic acid includes three or more separate polynucleotides.

### Definitions

The following presents definitions of some terms presented throughout this disclosure. In some instances, terms are defined in areas of this specification other than in this “Definitions” section.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, and more preferably within 2-fold, of a value. Where particular values are described in the application and claims, unless otherwise stated, the term “about” means within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

An article, composition, method, or the like that comprises one or more elements may consist of the one or more elements or may consist essentially of the one or more elements. As used in this specification and claim(s), “consisting of” (and any form of consisting of, such as “consists of” and “consist of”) means including and limited to. As used in this specification and claim(s), an article, composition, method, or the like “consisting essentially of” (and any form of consisting essentially of, such as “consists essentially of” and “consist essentially of”) means the article, composition, method, or the like includes the specified enumerated elements; such as components, compounds, materials, steps, or the like, and may include additional elements that do not materially affect the basic and novel characteristics of the article, composition, method, or the like.

Reference in the specification to “some embodiments,” “an embodiment,” “one embodiment,” “one or more embodiments,” “embodiments” or “other embodiments” means that a particular feature, structure, or characteristic described in connection with the embodiments is included in at least one or more embodiments, but not necessarily all embodiments, of the present disclosure.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The term “nucleic acid” as used herein refers to a polymer containing at least two nucleotides (i.e., deoxyribonucleotides or ribonucleotides) in either single- or double-stranded form and includes DNA and RNA. “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. Nucleic acids include nucleic acids containing known nucleotide analogs or modified backbone residues or linkages or modified sugar residues, or non-canonical/chemically-modified nucleobases and combinations thereof, which are synthetic, naturally occurring, and non-naturally occurring, and which have similar binding properties as the reference nucleic acid. Examples of such analogs and/or modified residues include, without limitation, phosphorothioates, phosphoramidates, methyl phosphonates, chiral-methyl phosphonates, 2′-O-methyl ribonucleotides, and peptide-nucleic acids (PNAs).

The term “nucleic acid” includes any oligonucleotide or polynucleotide, with fragments containing up to 60 nucleotides generally termed oligonucleotides, and longer fragments termed polynucleotides. A deoxyribooligonucleotide consists of a 5-carbon sugar called deoxyribose joined covalently to phosphate at the 5′ and 3′ carbons of this sugar to form an alternating, unbranched polymer. DNA may be in the form of, for example, antisense molecules, plasmid DNA, pre-condensed DNA, a PCR product, vectors, expression cassettes, chimeric sequences, chromosomal DNA, or derivatives and combinations of these groups. A ribooligonucleotide consists of a similar repeating structure where the 5-carbon sugar is ribose. Accordingly, the terms “polynucleotide” and “oligonucleotide” can refer to a polymer or oligomer of nucleotide or nucleoside monomers consisting of naturally-occurring bases, sugars and intersugar (backbone) linkages. The terms “polynucleotide” and “oligonucleotide” can also include polymers or oligomers comprising non-naturally occurring monomers, or portions thereof, which function similarly. Such modified or substituted oligonucleotides are often preferred over native forms because of properties such as, for example, enhanced cellular uptake, reduced immunogenicity, and increased stability in the presence of nucleases. It should be understood that the terms “polynucleotide” and “oligonucleotide” can also include polymers or oligomers comprising both deoxy and ribonucleotide combinations or variants thereof in combination with backbone modifications, such as those described herein.

The “nucleic acid” described herein may include one or more nucleotide variants, including nonstandard nucleotide(s), non-natural nucleotide(s), nucleotide analog(s), and/or modified nucleotides. Examples of modified nucleotides include, but are not limited to diaminopurine, 5-fluorouracil, 5-bromouracil, 5-chlorouracil, 5-iodouracil, hypoxanthine, xantine, 4-acetyl cytosine, 5-(carboxyhydroxylmethyl)uracil, 5-carboxymethylaminomethyl-2-thiouridine, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyl adenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl) uracil, (acp3)w, 2,6-diaminopurine and the like. In some cases, nucleotides may include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates).

The nucleic acid described herein may be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety, or phosphate backbone. Backbone modifications can include, but are not limited to, a phosphorothioate, a phosphorodithioate, a phosphoroselenoate, a phosphorodiselenoate, a phosphoroanilothioate, a phosphoraniladate, a phosphoramidate, and a phosphorodiamidate linkage. A phosphorothioate linkage substitutes a sulfur atom for a non-bridging oxygen in the phosphate backbone and delay nuclease degradation of oligonucleotides. A phosphorodiamidate linkage (N3′→P5′) prevents nuclease recognition and degradation. Backbone modifications can also include peptide bonds instead of phosphorous in the backbone structure (e.g., N-(2-aminoethyl)-glycine units linked by peptide bonds in a peptide nucleic acid), or linking groups including carbamate, amides, and linear and cyclic hydrocarbon groups. Oligonucleotides with modified backbones are reviewed in Micklefield, Curr. Med. Chem., 8 (10): 1157-79, 2001 and Lyer et al., Curr. Opin. Mol. Ther., 1 (3): 344-358, 1999. Nucleic acid molecules described herein may contain a sugar moiety that comprises ribose or deoxyribose, as present in naturally occurring nucleotides, or a modified sugar moiety or sugar analog. Modified sugar moieties include, but are not limited to, 2′-O-methyl, 2′-O-methoxyethyl, 2′-O-aminoethyl, 2′-Fluoro, N3′→P5′ phosphoramidate, 2′ dimethylaminooxyethoxy, 2′ 2′dimethylaminoethoxyethoxy, 2′-guanidinium, 2′-O-guanidinium ethyl, carbamate modified sugars, and bicyclic modified sugars. 2′-O-methyl or 2′-O-methoxyethyl modifications may be included to promote the “A-form” or “RNA-like” conformation in oligonucleotides, increase binding affinity to RNA, and enhance nuclease resistance. Modified sugar moieties can also include an extra bridge bond (e.g., a methylene bridge joining the 2′-O and 4′-C atoms of the ribose in a locked nucleic acid) or sugar analog such as a morpholine ring (e.g., as in a phosphorodiamidate morpholino).

The present disclosure encompasses isolated or substantially purified nucleic acid molecules and compositions containing those molecules. As used herein, an “isolated” or “purified” DNA molecule or RNA molecule is a DNA molecule or RNA molecule that exists apart from its native environment. An isolated DNA molecule or RNA molecule may exist in a purified form or may exist in a non-native environment such as, for example, a transgenic host cell. For example, an “isolated” or “purified” nucleic acid molecule or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived.

As used herein, the terms “protein,” “polypeptide,” and “peptide” are used interchangeably and refer to a polymer of amino acid residues linked via peptide bonds and which may be composed of two or more polypeptide chains. The terms “polypeptide,” “protein,” and “peptide” refer to a polymer of at least two amino acid monomers joined together through amide bonds. An amino acid may be the L-optical isomer or the D-optical isomer. More specifically, the terms “polypeptide,” “protein,” and “peptide” refer to a molecule composed of two or more amino acids in a specific order; for example, the order as determined by the base sequence of nucleotides in the gene or RNA coding for the protein. Proteins are essential for the structure, function, and regulation of the body's cells, tissues, and organs, and each protein has unique functions. Examples of proteins include hormones, enzymes, antibodies, and any fragments thereof. In some cases, a protein can be a portion of the protein, for example, a domain, a subdomain, or a motif of the protein. In some cases, a protein can be a variant (or mutant) of the protein, wherein one or more amino acid residues are inserted into, deleted from, and/or substituted into the naturally occurring (or at least a known) amino acid sequence of the protein. A protein or a variant thereof can be naturally occurring or recombinant. Methods for detection and/or measurement of polypeptides in biological material are well known in the art and include, but are not limited to, Western blotting, flow cytometry, enzyme-linked immunosorbent assay (ELISA), radioimmunoassay (RIA), and various proteomics techniques, such as mass spectrometry. An exemplary method to measure or detect a polypeptide is an immunoassay, such as an ELISA. This type of protein quantitation can be based on an antibody capable of capturing a specific antigen, and a second antibody capable of detecting the captured antigen.

Ranges provided herein are understood to be shorthand for all the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

Numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The term “complementary” is used throughout this application to describe two related nucleic acid sequences that may form a double-stranded complex of a first 5′ to 3′ “top” strand and a second 3′ to 5′ “bottom” strand. A spacer sequence of a guide nucleic acid is “complementary” to a target sequence on a strand opposite a protospacer sequence of a target nucleic acid. In the context of guide nucleic acid, a sequence may be considered sufficiently “complementary” to a target sequence if it may be used to guide an editor protein to the target sequence to cause an intended edit.

A spacer sequence of a guide nucleic acid is considered to be “homologous” to a protospacer sequence of a target nucleic acid. A first sequence that is homologous to a second sequence may be identical to or substantially identical to the second sequence. In the context of gRNA, a spacer sequence may be considered “homologous” to the protospacer sequence if the gRNA may be used to guide an editor protein to the target sequence to cause an intended edit. A spacer sequence that is homologous to a protospacer sequence may be identical or substantially identical to the protospacer sequence.

As used herein, a nucleic acid sequence that is “substantially identical” to another nucleic acid sequence is a nucleotide sequence that has 70% or more sequence identity to the other nucleic acid sequence, such as 75% or more sequence identity, 80% or more sequence identity, 85% or more sequence identity, 90% or more sequence identity, 95% or more sequence identity, 96% or more sequence identity, 97% or more sequence identity, 98% or more sequence identity, or 99% or more sequence identity to the other nucleic acid sequence.

For purposes of percent sequence identity between an RNA sequence and a DNA sequence, uracil bases in the RNA are to be considered identical to thymine bases in DNA sequences.

As used herein “sequence identity” refers to the extent to which two optimally aligned nucleic acid sequences are invariant throughout a window of alignment of components, e.g., nucleotides. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) nucleic acid (or its complementary strand) as compared to a test (“subject”) nucleic acid (or its complementary strand) when the two sequences are optimally aligned. Percent sequence identity may be determined, when the compared sequences are aligned for maximum correspondence, as measured using a sequence comparison algorithm described below and as known in the art, or by visual inspection.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters. Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). Alignment of sequences may be analyzed using a Burrows-Wheeler transform such as BOWTIE open-source software available from https://github.com/BenLangmead/bowtie. An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100.

“Percent identity” may also be determined using BLASTX version 2.0 for translated nucleotide sequences and BLASTN version 2.0 for polynucleotide sequences. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands.

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleotide sequence to the reference nucleotide sequence is less than about 0.1 to less than about 0.001.

“Guide nucleic acid”, “guide RNA”, and “gRNA” are used interchangeably herein. A gRNA may comprise one or more DNA nucleotides and/or one or more modified nucleotides.

The term “subject” or “patient” encompasses mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like.

“A subject in need thereof” refers to an individual who has a disease, a symptom of the disease, or a predisposition toward the disease, with the purpose to cure, heal, alleviate, relieve, alter, remedy, ameliorate, improve, or affect the disease, the symptom of the disease, or the predisposition toward the disease. In one or more embodiments, the subject has cardiovascular disease due at least in part to an elevated blood Lp(a) concentration.

“Administering” and its grammatical equivalents as used herein can refer to providing one or more drug substances (e.g., mRNA that encodes the editor proteins, guide molecules), drug products (e.g., LNPs that encapsulate the drug substances for delivery to the target cells/tissue) or pharmaceutical compositions thereof as described herein to a subject or a patient. By way of example and without limitation, “administering” can be performed by intravenous (i.v.) injection, sub-cutaneous (s.c.) injection, intradermal (i.d.) injection, intraperitoneal (i.p.) injection, intramuscular (i.m.) injection, intravascular injection, intracerebroventricular (i.c.v.) injection, intrathecal (i.t.) injection, infusion (inf.), oral routes (p.o.), topical (top.) administration, or rectal (p.r.) administration. One or more such routes can be employed.

The term “parenteral” as used herein includes subcutaneous, intracutaneous, intravenous, intramuscular, intraarticular, intraarterial, intrasynovial, intrasternal, intracerebroventricular, intrathecal, intralesional, and intracranial injection or infusion techniques. Parenteral administration can be, for example, by bolus injection or by gradual perfusion over time. In addition, it can be administered to the subject via injectable depot routes of administration such as using 1-, 3-, or 6-month depot injectable or biodegradable materials and methods.

The terms “treat,” “treating,” or “treatment,” and its grammatical equivalents as used herein, can include alleviating, abating, or ameliorating at least one symptom of a disease or a condition, preventing additional symptoms, inhibiting the disease or the condition, e.g., arresting the development of the disease or the condition, relieving the disease or the condition, causing regression of the disease or the condition, relieving a condition caused by the disease or the condition, or stopping the symptoms of the disease or the condition either prophylactically and/or therapeutically. “Treating” may refer to administration of a composition comprising a nanoparticle, such as a lipid nanoparticle (LNP), to a subject before or after the onset, or suspected onset, of a disease or condition. “Treating” includes the concepts of “alleviating,” which refers to lessening the frequency of occurrence or recurrence, or the severity, of any symptoms or other ill effects related to a disease or condition and/or the side effects associated with the disease or condition. The term “treating” also encompasses the concept of “managing” which refers to reducing the severity of a particular disease or disorder in a patient or delaying its recurrence, e.g., lengthening the period of remission in a patient who had suffered from the disease. The term “treating” further encompasses the concept of “prevent,” “preventing,” and “prevention.” It is appreciated that, although not precluded, treating a disorder or condition does not require that the disorder, condition, or symptoms associated therewith be completely eliminated.

As used herein, the terms “prevent,” “preventing,” “prevention,” and the like, refer to reducing the probability of developing a disease or condition in a subject, who does not have, but is at risk of or susceptible to developing a disease or condition.

The term “ameliorate” as used herein can refer to decrease, suppress, attenuate, diminish, arrest, or stabilize the development or progression of a disease.

As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset.

As used herein “onset” or “occurrence” of a disease includes initial onset and/or recurrence.

The term “therapeutic agent” or “drug substance” can refer to any agent that, when administered to a subject, has a therapeutic, diagnostic, and/or prophylactic effect and/or elicits a desired biological and/or pharmacological effect. Therapeutic agents can also be referred to as “actives” or “active agents.” Such agents include, but are not limited to, cytotoxins, radioactive ions, chemotherapeutic agents, small molecule drugs, proteins, and nucleic acids such as guide molecules and mRNA.

The terms “pharmaceutical composition” and its grammatical equivalents as used herein can refer to a mixture or solution comprising a therapeutically effective amount of an active pharmaceutical ingredient together with one or more pharmaceutically acceptable excipients, carriers, and/or a therapeutic agent to be administered to a subject, e.g., a human in need thereof.

The term “pharmaceutically acceptable” and its grammatical equivalents as used herein can refer to an attribute of a material which is useful in preparing a pharmaceutical composition that is generally safe, non-toxic, and neither biologically nor otherwise undesirable and is acceptable for veterinary as well as human pharmaceutical use. “Pharmaceutically acceptable” can refer to a material, such as a carrier or diluent, which does not abrogate the biological activity or properties of the compound, and is relatively nontoxic, i.e., the material may be administered to a subject without causing undesirable biological effects or interacting in a deleterious manner with any of the components of the pharmaceutical composition in which it is contained.

A “pharmaceutically acceptable excipient, carrier, or diluent” refers to an excipient, carrier, or diluent that can be administered to a subject, together with an agent, and which does not destroy the pharmacological activity thereof and is nontoxic when administered in doses sufficient to deliver a therapeutic amount of the agent.

A “pharmaceutically acceptable salt” may be an acid or base salt that is generally considered in the art to be suitable for use in contact with the tissues of human beings or animals without excessive toxicity, irritation, allergic response, or other problem or complication. Those of ordinary skill in the art will recognize from this disclosure and the knowledge in the art that further pharmaceutically acceptable salts include those listed by Remington's Pharmaceutical Sciences, 17th ed., Mack Publishing Company, Easton, PA, p. 1418 (1985).

As used herein, the term “therapeutically effective amount” means an amount of an agent to be delivered (e.g., nucleic acid, drug, payload, composition, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Ranges provided herein are understood to be shorthand for all the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to and 50 to 10 in the other direction.

In several places throughout the application, guidance is provided through examples, which examples, including the particular aspects thereof, can be used in various combinations and be the subject of claims. In each instance, the recited list serves only as a representative group and should not be interpreted as an exclusive list. It is to be understood that the particular examples, materials, amounts, and procedures are to be interpreted broadly in accordance with the scope and spirit of the invention as set forth herein.

For any method disclosed herein that includes discrete steps, the steps may be conducted in any feasible order. And, as appropriate, any combination of two or more steps may be conducted simultaneously.

All headings throughout are for the convenience of the reader and should not be used to limit the meaning of the text that follows the heading, unless so specified.

### Gene Editor Systems

Gene editor systems have the potential to transform modern medicine by offering in vivo and ex vivo treatments and cures for genetic diseases. Gene editor systems typically include guide nucleic acid molecules and gene editors. Gene editors comprise one or more proteins that carry out an intended edit in the presence of a guide nucleic acid. For example, a gene editor may comprise DNA binding domain and an editing domain. In embodiments, gene editors include fusion proteins. For example, a gene editor may comprise a nucleotide-directed DNA binding domain fused to an editing domain. In embodiments, gene editors include more than one protein, which are not fused. For example, a gene editor may comprise a first protein having nucleotide-directed DNA binding activity and a separate second protein having editing activity. One or more of the activities of the separate domains or proteins may be coordinated by the guide nucleic acid.

At its most basic, a guide nucleic acid includes elements to (i) bind a gene editor and (ii) guide the editor to a desired location of a gene. A guide nucleic acid may include additional elements, such as an editing template and/or an RNA aptamer. A guide nucleic acid may be designed to include combinations of guide nucleic acid elements to improve properties such as specificity, efficiency, and stability, as well as compatibility with a particular gene editor.

A guide nucleic acid typically includes regions with specific secondary structure, such as a general lack of secondary structure (e.g., linear), stem-loop structures, or the like. For example, a spacer element of a guide nucleic acid typically is linear, while a guide nucleic acid element that binds a gene editor typically comprises one or more stem-loop structures. A guide nucleic acid may be formed from one or more polynucleotides that, when assembled, include proper secondary structure and tertiary structure to cause a gene editor to carry out an intended edit.

Most artificial guide nucleic acids are chemically synthesized as a single continuous sequence (also referred to as “single-guide nucleic acid,” “single guide RNA,” “sg nucleic acid,” or “sgRNA”), which contrasts with naturally occurring two-piece guide RNA complexes. Single guide nucleic acids provide advantages over two-piece guide RNA complexes in that they may more readily form proper secondary structure and tertiary structure, and they may be more simple or cost-effective to synthesize.

Artificial guide nucleic acids are typically chemically synthesized using reactions that involve adding one nucleotide at a time to a growing polynucleotide. While the likelihood that the correct nucleotide is added at each step of the process is quite high, as the length of the polynucleotide increases, the likelihood that the entire sequence of nucleotides in the nucleic acid are correct can be substantially lower. Accordingly, the quality and uniformity of chemically synthesized polynucleotides, such as gRNA, generally decreases with increasing length. It is desirable to engineer guide nucleic acid designs that minimize the length of synthesized polynucleotides while maximizing the possibility of modularly by combining guide nucleic acid elements.

In embodiments described herein, guide nucleic acids include two, three, or more polynucleotides, each including a guide nucleic acid element, or a portion thereof, and one or more hybridization sequences. The guide nucleic acids described herein are compatible with assembly of guide nucleic acid libraries for testing many different sequences and designs in parallel. The designs described herein may additionally have higher sequence fidelity than sgRNAs, because each polynucleotide of the guide nucleic acids described herein may have lengths that are shorter than single guide nucleic acids.

### Template-Based Gene Editors

The hybridized guide nucleic acids described herein may be compatible with any suitable template-based gene editor. A template-based editor is one or more protein capable of writing new genetic information into a targeted DNA site using a portion of a guide nucleic acid as a template. The portion of the guide nucleic acid that is used as the template is referred to herein as the “editing template.” A template-based gene editor may comprise a domain or protein having editing activity. The domain or protein having editing activity may have RNA-dependent DNA polymerase (e.g., reverse transcriptase) activity if the editing template is an RNA editing template. The domain or protein having editing activity may have DNA-dependent DNA polymerase activity if the editing template is a DNA editing template. Examples of suitable DNA polymerase proteins or domains for use in template-based editors include those described in, for example, Halperin et al., “CRISPR-guided DNA polymerases enable diversification of all nucleotides in a tunable window,” Nature, 2018 Aug. 1, 560, 248-252

A template-based gene editor may comprise a domain or protein having DNA binding activity. In some embodiments, the domain or protein having DNA binding activity comprises a zinc-finger element or a TAL effector element, e.g., a zinc-finger or TAL polypeptide or functional fragment thereof. In some embodiments, the domain or protein having DNA binding activity comprises a sequence-guided DNA binding element, such as Cas9, Cpfl, or other CRISPR-related protein. The sequence-guided DNA binding element may be catalytically inactivated or impaired such that it does not cut a single-stranded nucleic acid target or such that it nicks or cuts at most one strand of a double-stranded nucleic acid target. In some embodiments, the domain or protein having DNA binding activity comprises one or more of Cas9, TAL domain, ZF domain, Myb domain, combinations thereof, or multiples thereof. In some embodiments, the domain or protein having DNA binding activity comprises a DNA binding domain of a retrotransposon described in U.S. Patent Application Publication No. 2020/0109398 A1, entitled METHODS AND COMPOSITIONS FOR MODULATING A GENOME. In some embodiments, the domain or protein having DNA binding activity comprises a Cas-type domain or protein, such as Cas9. Examples of Cas-type domains or proteins that may be suitable for use with the hybridized guide nucleic acids described herein include those disclosed by Cong et al., Science 2013, 339, 819-823, Mali et al. Science 2013, 339, 823-826, Hwang et al., Nature Biotechnology 2013, 31, 227-229; Jinek et al., eLife 2013 2, e00471, Dicarlo et al. Nucleic Acids Research 2013; Jiang et al., Nature Biotechnology 2013 31, 233-239, and Jinek et al. Science 2012 Aug. 17; 337(6096):816-21.

A template-based editor may include any other suitable domain or protein having any other suitable activity. For example, the template-based editor may comprise an endonuclease or an endonuclease domain. The endonuclease or endonuclease domain may be a retrotransposon endonuclease. The endonuclease or endonuclease domain may be a retrotransposon endonuclease/DNA binding domain protein or domain. Examples of retrotransposon endonuclease or endonuclease/DNA binding proteins or domains are described in, for example, U.S. Patent Application Publication No. 2020/0109398 A1, entitled METHODS AND COMPOSITIONS FOR MODULATING A GENOME.

In some embodiments, a template-based gene editor system (the gene editor and one or more guide nucleic acids) is capable of locating a specific target in a gene or genome, cutting the phosphate backbone of the target sequence, and introducing a specific gene edit to the target sequence via an editing template. A template-based editor may comprise a nucleic acid binding domain that can be programmed to bind a specific nucleic acid sequence. The nucleic acid binding domain may be catalytically inactivated or impaired such that it does not cut a single-stranded nucleic acid target or such that it nicks or cuts at most one strand of a double-stranded nucleic acid target.

In some embodiments, a template-based gene editor includes a protein comprising a nucleotide-directed DNA binding domain fused or linked to an editing domain. The editing domain may have polymerase activity, such as reverse transcriptase activity. A template-based editor may include one or more linkers, for example, peptide linkers between domains. The nucleotide-directed DNA binding domain may be Cas9 or a Cas9-based protein, such as a nickase Cas9, a catalytically dead Cas9, or a Cas9 that has been otherwise engineered.

In some embodiments, the template-based gene editor comprises a protein or domain comprising reverse transcriptase activity. One example of a template-based gene editor having reverse transcriptase activity is prime editor. Prime editor is a template-based gene editor initially described in 2019 (Anzalone et al. Nature 2019 December; 576(7785)). Prime editor systems generally include a nickase Cas9 and a Moloney murine leukemia virus reverse transcriptase (M-MLV RT). The guide nucleic acid typically used in prime editing systems is referred to as a “prime editing guide RNA” (pegRNA) and contains an editing template. The pegRNA also includes a primer-binding site (PBS). The PBS may be designed to hybridize with the displaced strand on the 5′ side of the introduced cut. The PBS may be complementary to a portion of the protospacer sequence. The editing template sequence includes the edit to be installed and is typically located between the tracr region and the primer binding site. The length of the edit to be installed may vary, from deletions of 10 or fewer nucleotides to insertions of more than 80 nucleotides.

Template-based gene editors that may be compatible with the hybridized guide nucleic acid designs described herein include those described in Anzalone et al. Nature 2019 December; 576(7785), Chen et al. Cell 2021 Oct. 28; 184(22):5635-5652.e29, Nelson et al. Nat. Biotechnol. 2022 March; 40(3):402-410, Reint, Ganna et al. eLife 2021, “Rapid genome editing by CRISPR-Cas9-POLD3 fusion.” Vol. 10 e75415. 13, Liu, Bin et al. Nat biotechnol 2022 “A split prime editor with untethered reverse transcriptase and circular RNA template.” 10.1038/s41587-022-01255-9, and Wang, Jue et al. Yi Chuan 2019 vol. 41,5: 422-429.

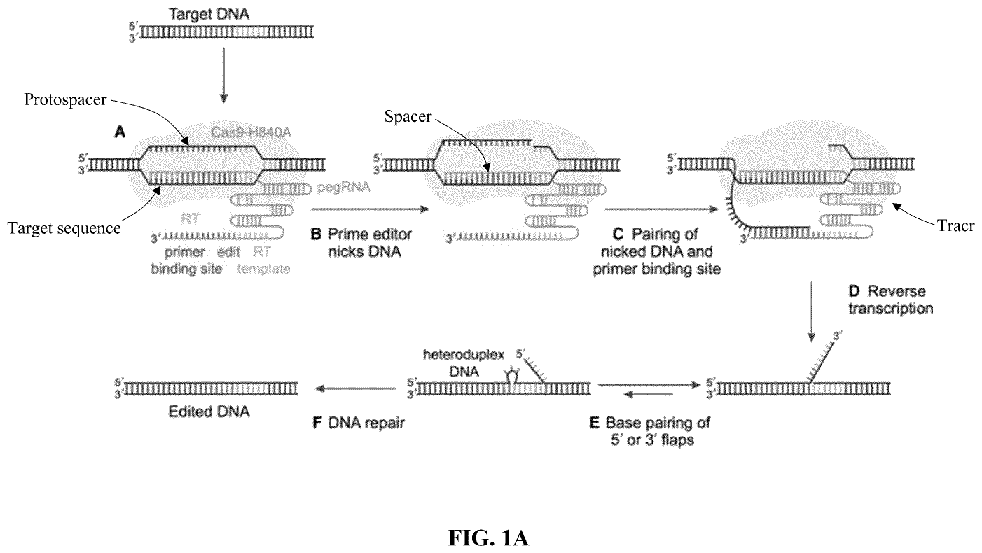

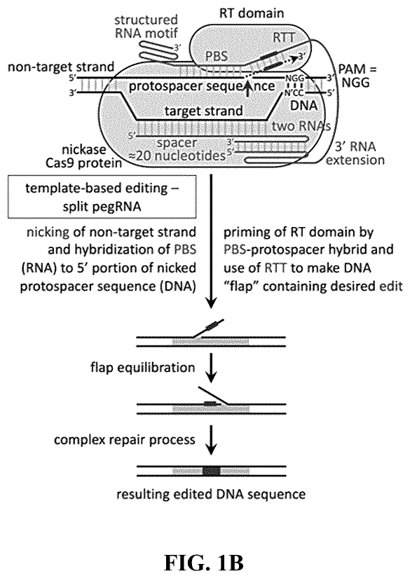

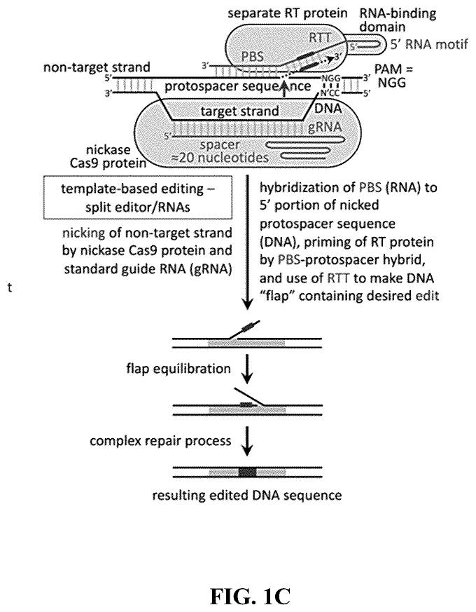

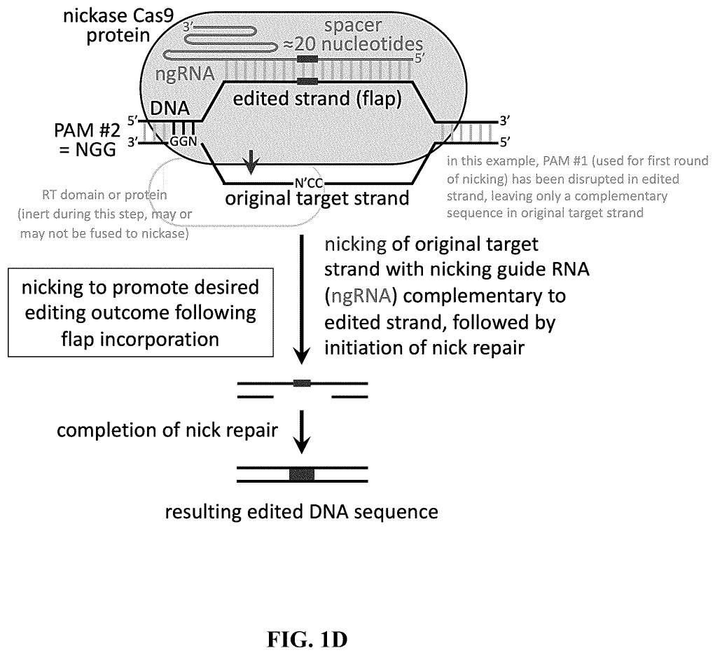

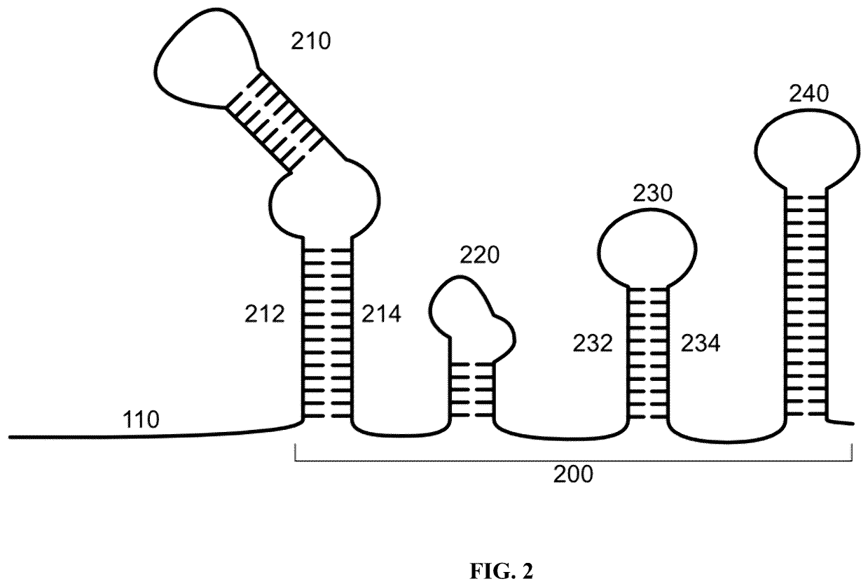

For purposes of general illustration, FIG. 1A shows steps associated with editing using an embodiment of a template-based editing system. In the depicted embodiment, the target sequence on the strand opposite the protospacer is bound by the Cas9-H840A domain via the spacer region of the guide RNA (pegRNA) (A). The Cas9-H840A domain cuts the displaced strand (B). The displaced strand then may pair with the primer binding site (PBS) (C). The RT may recognize the RNA-DNA duplex formed by the displaced strand and PBS and extend the DNA of the displaced strand in the 3′ direction, using the editing template (RT template) of the pegRNA as a template (D). This creates a “flap” of single-stranded DNA on the displaced strand including the desired edit. The prime editor may then dissociate from the DNA, leaving two redundant “flaps” on the displaced strand, wherein one flap is the original sequence, and one flap is the edited sequence. Through a process called “flap equilibration”, one of the sequences will bind the target sequence, and the other will remain attached to the displaced strand as a single-stranded flap (E). If the flap with the edited sequence is bound by the target sequence, the complex may be called a “DNA heteroduplex”, referring to the mismatch caused by the edit. Cellular DNA repair machinery may then act on the DNA heteroduplex, incorporating the edit (F).

FIG. 1B illustrates a split-guide form of template-based editing comprising a nickase Cas9 protein fused to a reverse transcriptase domain and that is operationally assembled with a hybridized having two portions that hybridized to form a guide nucleic acid. One portion of the hybridized guide nucleic acid comprises the spacer and at least a portion of the tracr region of the gRNA which interacts with the Cas9 nickase to facilitate nicking within the protospacer sequence. The other portion of the hybridized guide nucleic acid comprises the remaining portion of the pegRNA sequence that includes the PBS and RTT regions, which allows the reverse transcriptase domain to insert the desired edit into the nicked strand of the targeted DNA site.

FIG. 1C illustrates split editor and split guide form of template-based editing comprising a nickase Cas9 protein that has assembled with a first gRNA comprising the spacer and full tracr region to facilitate nicking within the protospacer sequence in the targeted DNA site, and a separate reverse transcriptase protein fused to an RNA-binding domain that is operationally interactive with a second guide that comprises the PBS, the RTT, and an RNA motif that binds to the RNA-binding domain, which inserts the desired edit into the nicked strand of the targeted DNA site. The RNA motif may be a MS2 (based on the MS2 bacteriophage coat protein) motif and the RNA-binding domain may be a bacteriophage coat protein (MCP) domain.

Hybridized guides as described herein may operate according to the mechanism shown in FIG. 1C with a split editor to effectively produce gene edits. The location of the RNA motif may on one or more of the polynucleotide components of the hybridized guide may impact editing efficiency.

FIG. 1D is a schematic drawing illustrating the use of a nicking guide RNA (ngRNA) after the initial template-based editing, as illustrated in, for example, FIG. 1A, FIG. 1B, or FIG. 1C, has occurred. Upon disassembly of the gene editor assembly detailed in FIGS. 1A-C from the targeted DNA site and incorporation of the newly synthesized flap into the double helix at the targeted DNA site, the edited strand that has incorporated the flap is mismatched to the opposite strand, which retains the original gene sequence. Addition of a ngRNA allows for the assembly of the gene editor with the ngRNA, which is complementary (or otherwise designed to hybridize) to the edited strand in the portion directly 5′ of a PAM, to effect a nick in the original target strand, which instigates a nick repair mechanism of the cell that results in replacement of the sequence in the original strand around the nick with a sequence harboring the desired edit. The nickase Cas9 protein shown in FIG. 1D may be a nickase component or domain of a gene editor system illustrated in any one of FIG. 1A-C or may be a separate protein independent of the gene editor systems illustrated in FIGS. 1A-C.

### Guide Nucleic Acids

The structure of guide nucleic acids designed and synthesized for therapeutic use may differ from the structure of naturally occurring gRNA. In part, the structure of guide nucleic acids for therapeutic use is different because they are targeted to particular gene locations, because they are designed to install particular edits, and/or because they are designed to be compatible with a particular gene editor. Gene editors designed for therapeutic use may be modified relative to naturally occurring gene editors, and thus guide nucleic acids for use with modified gene editors may be modified relative to naturally occurring guide nucleic acids to be compatible with the modified gene editors.

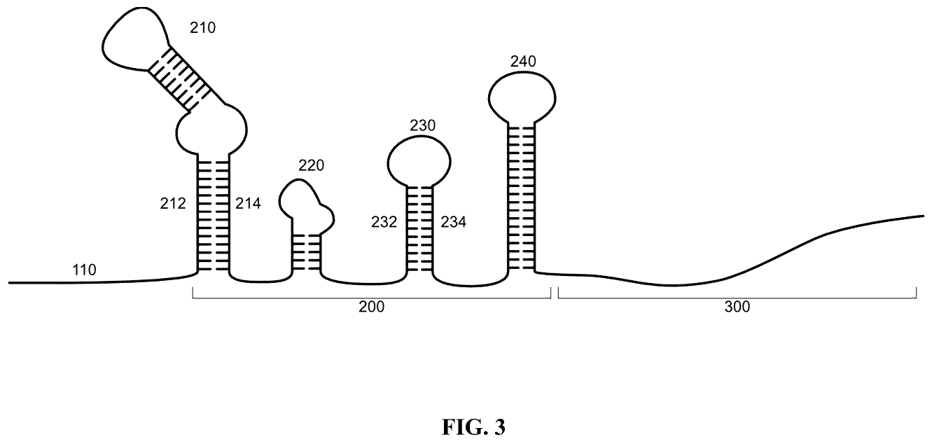

The structure of guide nucleic acids designed and synthesized for therapeutic use may also differ from the structure of naturally occurring gRNAs, as the structure of guide nucleic acids for therapeutic use may include single guide nucleic acids, such as sgRNAs. Schematic examples of single guide nucleic acids that have been designed for therapeutic use are shown in FIGS. 2-3. In FIGS. 2-3, the guide nucleic acid includes a spacer region 110 and a tracr region 200. The spacer region 110 is typically at, or in proximity to, the 5′ end of the guide nucleic acid. The spacer 110 is complementary to a target sequence (e.g., a sequence complementary to a protospacer sequence) in a target chromosomal site and may serve to guide a gene editor to the target site. The tracr region 200 may complex with, or “bind,” a gene editor. The tracr region 200 is typically 3′ relative to the spacer region 110. The tracr region 110 may form well defined secondary and tertiary structure that is recognized by the gene editor. For example, the tracr region may form one or more stem-loop structures (components labeled as 210, 212, 214, 220, 230, 232, 234, and 240). As shown in FIG. 3, a guide nucleic acid, such as a gRNA, may include one or more additional region 300, which is depicted as a 3′ extension. In the embodiment depicted in FIG. 3, the additional region 300 is linear (e.g., generally free of stem-loop structures with the region itself). However, a portion of the additional region 300 may hybridize (not shown) to a portion of the spacer region 110. The additional region 300 may be configured for use with, for example, a template-based editor. A gRNA configured for use with a template-based editor may be referred to as a “pegRNA”.

In embodiments (not shown), a guide nucleic acid may include additional motifs. The additional motifs may be, for example, on the 3′ end. The additional motifs may serve any suitable purpose. For example, the additional motifs may serve to provide suitable space between elements, may improve stability of the guide nucleic acid, may improve affinity for a gene editor, and combinations thereof, or the like.

The spacer region, tracr region, editing template, and other motifs of a guide nucleic acid are “guide nucleic acid elements.” A guide nucleic acid element is a portion of the guide nucleic acid that is required for the guide nucleic acid to carry out a particular function. The primary sequence, secondary structure, and/or tertiary structure of the guide nucleic acid may determine whether the portion of the guide nucleic acid may carry out the particular function. Examples of functional elements of guide nucleic acids, such as gRNA elements, include a spacer region, a tracr region, an editing template, a primer binding site, additional motifs, and the like.

### Spacer Regions

In embodiments, a hybridized guide nucleic acid described herein includes a spacer region. The spacer region may be formed from one, or more than one, of the polynucleotides that are hybridized to form the guide nucleic acid. In embodiments, a single polynucleotide forms the entire spacer region. The hybridized guide nucleic acid may comprise any suitable spacer region. The spacer region comprises a sequence complementary to a target sequence at a target chromosomal location (e.g., a sequence complementary to a protospacer sequence). The sequence of the spacer region is dependent on the sequence of the protospacer.

The spacer region may comprise any suitable number of nucleotides. In embodiments, the spacer region is about 15 nucleotides to about 100 nucleotides long. In embodiments, at least 10 contiguous nucleotides of the spacer region are identical to a sequence of the protospacer. In embodiments, the spacer region is at the 5′ end of the guide nucleic acid. In embodiments, the spacer region is within 20 nucleotides, within 15 nucleotides, within 10 nucleotides, or within 5 nucleotides of the 5′ end of the guide nucleic acid. In embodiments, the 3′ end of the protospacer is immediately adjacent to a canonical S. pyogenes Cas9 PAM sequence (NGG). In some embodiments, the 3′ end of the protospacer sequence is not immediately adjacent to a canonical PAM sequence. In embodiments, the 3′ end of the protospacer sequence is immediately adjacent to an AGC, GAG, TTT, GTG, or CAA sequence.

Depending on the type of gene editor used, the PAM may vary, and/or the length of the spacer region may vary. In embodiments, the spacer region comprises 10 nucleotides to 60 nucleotides, 15 nucleotides to 40 nucleotides, or 20 nucleotides in length. For example, if S. aureus Cas9 is used, an NNGRRT PAM and a 17 to 26, such as 22-24, nucleotide spacer region sequence may be used. It should be apparent to one of skill in the art that optimal spacer region design may differ depending on the gene editor used. The guide nucleic acids described herein may be compatible with many different gene editors and may be used in combination with many different PAM sequences, which may vary depending on the gene editor.

### Tracr Regions

In embodiments, a hybridized guide nucleic acid described herein includes a tracr region. The tracr region may be formed from one, or more than one, of the polynucleotides that are hybridized to form the guide nucleic acid. The hybridized guide nucleic acid may comprise any suitable tracr region. The sequence of the tracr region is dependent on the gene editor with which the guide nucleic acid is compatible. In embodiments, the tracr region forms a secondary and/or tertiary structure that is recognized by, and bound by, a compatible gene editor. The guide nucleic acids described herein may be compatible with many different gene editors and may be used in combination with many different tracr region sequences. The tracr region sequence included in the guide nucleic acids described in this disclosure may be canonical to a gene editor, or it may be modified from a canonical sequence.

The tracr region may comprise any suitable nucleic acid sequence, may be of any suitable nucleotide length, and may be positioned in the guide nucleic acid at any suitable location. In some embodiments, the tracr region is positioned immediately adjacent the spacer region. In some embodiments, the tracr region is positioned within 20 nucleotides, within 15 nucleotides, within nucleotides, or within 5 nucleotides of the spacer region. In embodiments, the tracr region is positioned 3′ of the spacer region.

The tracr region may have any suitable length. In embodiments, the overall length of the tracr region is in a range from about 10 to about 2000 nucleotides in length. In embodiments, the tracr region may be at least 70, at least 80, or at least 90 nucleotides in length. In embodiments, the tracr region may be at most 200, at most 150 or at most 100 nucleotides in length. In some embodiments, the tracr region is 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 nucleotides in length.

The tracr region may include DNA nucleotides, RNA nucleotides, or a combination thereof. One or more nucleotides of a tracr region may be chemically modified. The tracr region may alternately not include any modified nucleotides.

In embodiments, the tracr region forms one or more base-paired stems. A base-paired stem may be part of a stem-loop structure. The base-paired stems may comprise stems of any suitable length. The loops of a stem-loop structure may be any suitable length. In embodiments, a portion of the guide nucleic acid folds back upon itself to form base pairs with nucleotides in another location of the guide nucleic acid to form the stem-loop structure. In some embodiments, a first polynucleotide of a hybridized guide nucleic acid hybridizes with a second polynucleotide of the hybridized nucleic acid to form a base-paired stem.

In some embodiments, all the nucleotides within a stem base pair with a corresponding nucleotide. In some embodiments, one or more nucleotides within the stem do not base pair with a corresponding nucleotide. For example, 1, 2, 3, 4, or 5 nucleotides with a stem do not base pair with a corresponding nucleotide.

In some embodiments, a strand of a base-paired stem is from about 5 to about 30 nucleotides in length, such as about 10 to about 20 nucleotides in length, or about 15 nucleotides in length.

In some embodiments, a loop of a stem-loop structure is from about 2 to about 20 nucleotides in length, such as from about 3 to about 15, or from about 4 to about 20 nucleotides in length.

Referring again to the embodiments depicted in FIGS. 2-3, the tracr region 200 comprises a stem-loop 210 including a first strand of the stem 212 and a second strand of the stem 214. As shown, the first 212 and second 214 strands of the stem are part of a single guide nucleic acid and are connected by a loop. However, each strand 212, 214 of the stem, or a portion thereof, may be formed from separate hybridized polynucleotides. When the stem-loop 210 is formed from separate polynucleotides, the stem-loop 210 may not be connected by a loop.

The tracr region 200 depicted in the embodiments of FIGS. 2-3 include additional portions with secondary structure, including a nexus 220, a first hairpin 230 and a second hairpin 240. Each hairpin 230, 240 includes two complementary regions and a loop. For example, the first hairpin 230 includes a first strand of the hairpin 232 and a second strand of the hairpin 234. As shown, the first strand 232 and second strand 234 of hairpin 230 are part of a single guide nucleic acid and are connected by a loop. However, each strand 232, 234 of the stem, or a portion thereof, may be formed from separate hybridized polynucleotides. Similarly, the stem of hairpin 240 may be formed from a single guide nucleic acid connected by a loop or may be formed from separate hybridized polynucleotides. When the hairpins 230, 240 are formed from separate polynucleotides, the hairpins 230, 240 may not be connected by a loop.

For purposes of the present disclosure, the complete tracr region (e.g, 200 as shown in FIGS. 2-3) may be referred to as a “guide nucleic acid element,” such as a “gRNA element.” One or more secondary structures (e.g., stem-loop 210, nexus 220, first hairpin 230, or second hairpin 240 as shown in FIGS. 2-3) may also be referred to herein as a “guide nucleic acid element”, such as a “gRNA element.”

### Editing Templates

In embodiments, a hybridized guide nucleic acid described herein includes an editing template. The editing template may be formed from one, or more than one, of the polynucleotides that are hybridized to form the guide nucleic acid. In embodiments, a single polynucleotide forms the entire editing template. The hybridized guide nucleic acid may comprise any suitable editing template. The sequence of the editing template is dependent on gene edit that is desired. The sequence of the editing template may be dependent on the chromosomal location in which the edit is inserted.

The editing template may comprise any suitable nucleic acid sequence, may be of any suitable nucleotide length, and may be positioned in the guide nucleic acid at any suitable location. In some embodiments, the editing template is positioned 3′ of the tracr region. In embodiments, the editing template is 1 to 200, 10 to 100, 20 to 80, or 60 nucleotides in length. In embodiments, the editing template is 1 to 100, 10 to 60, 15 to 50, or 30 nucleotides in length. In some embodiments, the editing template is 1, 2, 3, 4, 5, 6, 7, 8, 9, 10, 11, 12, 13, 14, 15, 16, 17, 18, 19, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 nucleotides in length.

The editing template may encode a desired edit to a gene. The desired edit may be an insertion, a deletion, or a substitution.

### Primer Binding Sites

In some embodiments, the hybridized guide nucleic acid includes a primer binding site (PBS). In embodiments, the PBS is 5 to 25, 10 to 20, or 12 to 15 nucleotides in length. In some embodiments, the PBS is 5, 6, 7, 8, 9, 10, 11, 12, 13, 14, 15, 16, 17, 18, 19, or 20 nucleotides in length. The PBS may be homologous to a region of a strand of genomic DNA into which the edit is inserted, and thus may be complementary to a region of a strand of genomic DNA. In embodiments, the PBS is complementary to at least a portion of the protospacer. The PBS may be complementary to a region of the protospacer adjacent to the location of a desired edit. In some embodiments, the 5′ most nucleotide of the PBS is complementary to the nucleotide at the 3′ end of the nick in the displaced strand.

In embodiments, a guide nucleic acid may include more than one editing template and associated PBS. Guide nucleic acids that have more than one editing template and associated PBS may exhibit higher rates of edit incorporation than guide nucleic acids that include only one editing template. The sequences of the multiple editing templates may be the same, or they may be different.

### Additional Motifs

In embodiments, a hybridized guide nucleic acid described herein includes one or more additional motifs. One or more of the additional motifs may be formed from one, or more than one, of the polynucleotides that are hybridized to form the guide nucleic acid. One or more of the additional motifs may confer properties that relate to editing. In embodiments, one or more of the additional motifs confer properties not directly related to editing. For example, one or more of the additional motifs may stabilize the guide nucleic acid, such as a gRNA, or prevent degradation by exonucleases. Examples of additional motifs that may be formed from one or more polynucleotides that are hybridized to form a guide nucleic acid described herein include an RNA aptamer, such as MS2 (based on the MS2 bacteriophage coat protein), F6, or PP7. Any suitable RNA aptamer may be included to confer desirable properties to the guide nucleic acid, such as a gRNA. In some embodiments, the motif may be a domain that increases affinity of the guide nucleic acid to the gene editor, such as an MCP domain. An additional motif may be positioned at any suitable location of the guide nucleic acid. For example, an additional motif may be on the 3′ end or the end of the guide nucleic acid, or there may be an addition motif on each of the 3′ end and the end. In some embodiments, the hybridized guide nucleic acid includes an RNA stabilizing motif such as a Mpknot motif, modified from a frameshifting pseudoknot from Moloney murine leukemia virus, or a TevoPreQ1 stabilizing motif.

Hybrid gRNA Design

Many different gRNA designs may be compatible with use with a given gene editor in a given context. The naturally occurring crRNA-tracrRNA complex is not often used in synthetic gene editor systems. sgRNA and pegRNA prepared from a contiguous single-stranded polynucleotide are typically used in synthetic gene editor systems. However, it may be desired to instead prepare a gRNA from multiple polynucleotides, each shorter than a corresponding single guide nucleic acid.

The guide nucleic acids described herein comprise two or more separate polynucleotides that form segments of the guide nucleic acid. The segments are designed such that a portion of one segment hybridizes to a portion of another segment. When hybridized or assembled, the segments form a functional guide nucleic acid that guides a gene editor to a target chromosomal location to install an intended edit in a target gene. In embodiments, at least one of the polynucleotides includes an editing template. In some embodiments, each polynucleotide segment includes at least one guide nucleic acid element. In some embodiments, one or more of the polynucleotide segments does not include a complete guide nucleic acid element but, when hybridized with another polynucleotide segment, forms a complete nucleic acid guide element.

In some embodiments, the hybridized guide nucleic acid includes more than one editing template. The hybridized guide may include a PBS for each editing template. In some embodiments, the hybridized guide nucleic acid comprises a first editing template and a second editing template, which may be the same or different. In embodiments, the guide nucleic acid includes a first editing template and a second editing template, wherein the first editing template and the second editing template are the same. In some embodiments, the sequence of the first editing template and sequence of the second editing template are at least 75%, at least 90%, or 100% identical. In some embodiments, the sequence of the second editing template are not substantially similar.

A hybridized guide nucleic acid may include two or more polynucleotides, each of which may include one or more guide nucleic acid elements, or a portion thereof, and one or more hybridization sequences. A guide nucleic acid element as described herein may be, or may be a portion of, a spacer region, a tracr region, an editing template, a primer binding site, an RNA motif, an RNA aptamer, or the like. In embodiments, a polynucleotide of a hybridized guide nucleic acid may include more than one guide nucleic acid element. In embodiments, a polynucleotide of a hybridized guide nucleic acid may include more than one hybridization sequence.

In some embodiments, a polynucleotide that forms a portion of a hybridized guide nucleic acid includes an entire guide nucleic acid element. In some embodiments, two or more polynucleotides form a portion of a hybridized guide nucleic acid assembly when hybridized to form a guide nucleic acid element.

In some embodiments, a hybridized guide nucleic acid includes two separate polynucleotide segments. In embodiments, a hybridized guide nucleic acid includes three separate polynucleotide segments. In embodiments, a hybridized guide nucleic acid includes four separate polynucleotide segments. In embodiments, a hybridized guide nucleic acid includes five separate polynucleotide segments.

A hybridized guide nucleic acid may be compatible with any suitable template-based gene editor. In embodiments, a hybridized guide nucleic acid described herein is compatible with a gene editor comprising a Cas9 or Cas9-related domain or protein or suitable variants of the domain or protein. In embodiments, the Cas9 or Cas9-related domain or protein comprises S. aureus Cas9. In embodiments, a guide nucleic acid described herein is compatible with a gene editor comprising a nickase Cas9 or a dead Cas9 protein or domain. In embodiments, a hybridized guide nucleic acid is compatible with a template-based editor. In embodiments, a hybridized guide nucleic acid is compatible with a composition of multiple template-based editors. In embodiments, a hybridized guide nucleic acid is compatible with a DNA-dependent DNA polymerase-based gene editor. In embodiments, a hybridized guide nucleic acid is compatible with a RNA-dependent DNA polymerase gene editor, such as a reverse transcriptase gene editor, such as a prime editor. In embodiments, a hybridized guide nucleic acid as described herein is compatible with a retrotransposon-based editor.

### Hybridization Sequences

In embodiments, each of the polynucleotides in the hybridized guide nucleotide includes one or more hybridization sequences to facilitate hybridization to another polynucleotide or other polynucleotides. The hybridization sequences may be complementary to each other. For example, in a hybridized guide nucleic acid including two polynucleotides, the first polynucleotide may include a first hybridization sequence that is complementary to a second hybridization sequence of the second polynucleotide. In a hybridized guide nucleic acid including three polynucleotides, the first polynucleotide may include a first hybridization sequence that is complementary to a second hybridization sequence of the second polynucleotide, and the second polynucleotide may further comprise a third hybridization sequence that is complementary to a fourth hybridization sequence of the third polynucleotide.

In embodiments, the hybridization sequences of polynucleotide segments are complementary over a length of 3 nucleotides or more, 5 nucleotides or more, 10 nucleotides or more, 15 nucleotides or more, 20 nucleotides or more, or 25 nucleotides or more. In embodiments, the hybridization sequences of polynucleotide segments are complementary over a length of 50 nucleotides or less, 40 nucleotides or less, or 30 nucleotides or less. In embodiments, the hybridization sequences of polynucleotide segments are complementary over a length of 3 to 50 nucleotides, such as 3 to 10 nucleotides, 4 to 9 nucleotides, 4 to 8 nucleotides, 10 to 40 nucleotides, or 15 to 30 nucleotides. In embodiments, the hybridization sequence of polynucleotide segments are complementary over a length of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides.

In embodiments, a hybridization sequence is on the 3′ end of the polynucleotide. In embodiments, the hybridization sequence is on the 5′ end of the polynucleotide. In embodiments where the polynucleotide includes more than one hybridization sequence, there may be one hybridization sequence on each of the 3′ and 5′ ends of the polynucleotide. In embodiments, the hybridization sequence may be positioned at a location that is not on either end of the polynucleotide.

In embodiments, the hybridization sequence of one polynucleotide forms a strand, or portion thereof, of a stem (e.g., of a stem-loop structure), and the complementary hybridization sequence of another polynucleotide forms the other strand, or portion thereof, of the stem.

In embodiments, the hybridization sequence of one polynucleotide forms a portion of a tracr region, and the complementary hybridization sequence of another polynucleotide forms a portion of the tracr region. In embodiments, the hybridization sequence of one polynucleotide forms a strand of a stem of the tracr region, and the complementary hybridization sequence of the other polynucleotide forms the other strand of the stem.

In embodiments, the hybridization sequence of a first polynucleotide forms a portion of the guide nucleic acid that serves to space apart elements of the guide nucleic acid, and the complementary hybridization sequence of a second polynucleotide hybridizes to the hybridization sequence of the first polynucleotide to form a double stranded portion that spaces apart elements of the guide nucleic acid.

In embodiments, the hybridization sequences comprise one or more modified nucleic acid. In embodiments, the modified nucleic acid includes a locked nucleic acid (LNA) or another modification that results in stronger binding to of the modified nucleotide to its complementary nucleotide relative to base pairing of natural base pairs.

### Modified Nucleotides

In embodiments, a hybridized guide nucleic acid includes DNA, RNA, and/or xenonucleic acid (XNA) nucleotides. The nucleotides of a hybridized guide nucleic acid may be chemically modified. In embodiments, the hybridization sequences of a hybridized guide nucleic acid are not chemically modified. A hybridized guide nucleic acid may include any nucleotides and modifications as described herein. For example, hybridized guide nucleic acid can be chemically modified to comprise a combination of 2′-O-methylribosugar and phosphorothioate backbone modifications on at least one 5′ nucleotide and at least one 3′ nucleotide of each hybridized guide nucleic acid polynucleotide. In some embodiments, the three terminal 5′ nucleotides and three terminal 3′ nucleotides of a polynucleotide are chemically modified to comprise combinations of 2′-O-methylribosugar and phosphorothioate modifications. In some embodiments, one or more polynucleotides of a hybridized gRNA may include these 5′ and 3′ modifications. In some other embodiments, the nucleotides of a hybridized guide nucleic acid may not be chemically modified

Library Assembly of gRNA

The polynucleotide segments that form the hybridized guide nucleic acids described herein may form libraries of polynucleotide segments that may be mixed and matched to provide guide nucleic acids that may target a particular chromosomal location (e.g., a location comprising a protospacer), provide compatibility with a particular gene editor, and/or cause the gene editor to install a particular edit.

### Design Options

In some embodiments, the hybridized guide nucleic acid includes an editing template. In some of these embodiments, the hybridized guide nucleic acid may be configured for use with a template-based gene editor, such as prime editor. In embodiments, a hybridized guide nucleic acid includes a first polynucleotide including a first guide nucleic acid element and a first hybridization sequence; and a second polynucleotide including a second guide nucleic acid element and a second hybridization sequence, wherein the second guide nucleic acid element comprises an editing template or an editing template and a PBS, wherein the first and second hybridization sequences are complementary, and wherein the first and second hybridization sequences are hybridized to one another. The editing template or editing template and PBS may have any suitable length as described herein. In some embodiments, both the first polynucleotide and the second polynucleotide may include an editing template or an editing template and a PBS.

In some embodiments, the hybridized guide nucleic acid includes chemically modified nucleotides. The chemical modifications may include any of those described herein. In embodiments, a hybridized guide nucleic acid includes a first polynucleotide, which may be chemically modified. In some of these embodiments, a second polynucleotide may also be chemically modified.

In some embodiments, the hybridized guide nucleic acid includes more than two polynucleotides. In embodiments, a hybridized guide nucleic acid includes a first polynucleotide including a first guide nucleic acid element and a first hybridization sequence; a second polynucleotide including a second guide nucleic acid element, a second hybridization sequence, and a third hybridization sequence; and a third polynucleotide comprising a third guide nucleic acid element and a fourth hybridization sequence, wherein the first and second hybridization sequences are complementary, and wherein the first and second hybridization sequences are hybridized to one another, wherein the third and fourth hybridization sequences are complementary, and wherein the third and fourth hybridization sequences are hybridized to one another.

In some embodiments, a hybridized guide nucleic acid may include more than three polynucleotides. In embodiments, a hybridized guide nucleic acid may include at least three, at least four, at least five, at least six, at least seven, at least eight, at least nine, or at least ten polynucleotides. In embodiments, a hybridized guide nucleic acid may include at most 20 polynucleotides.

In embodiments, a hybridized guide nucleic acid includes a plurality of polynucleotides; wherein each polynucleotide is at most 80 nucleotides in length, wherein each polynucleotide includes a guide nucleic acid element and a hybridization sequence, wherein each hybridization sequence is complementary to at least one other hybridization sequence, wherein at least one polynucleotide includes an editing template or an editing template and a PBS, and wherein the plurality of polynucleotides are hybridized to one another to form a hybridized guide nucleic acid.

In some embodiments, the hybridization sequence may include a portion of the tracr region sequence. In embodiments where the hybridized guide nucleic acid includes more than two polynucleotides, the hybridization sequence of each polynucleotide may include a portion of the tracr region sequence.

Referring again to the embodiments depicted in FIGS. 2-3, in some embodiments, the hybridization sequence may be or may include a portion of the tracr region. In embodiments where the tracr region is split, the hybridization sequence may include a portion of the tracr region sequence. For example, a first polynucleotide may have a hybridization sequence that includes the sequence of the first strand of the stem 212 and a second polynucleotide may have a hybridization sequence that includes the second strand of the stem 214. The first and second polynucleotides may be hybridized via the base pairing of the first and second strands of the stem 212 and 214. The hybridization sequence may include the first strand of a stem 212, the second strand of the stem 214, the first strand of the hairpin 232, or the second strand of the hairpin 232.

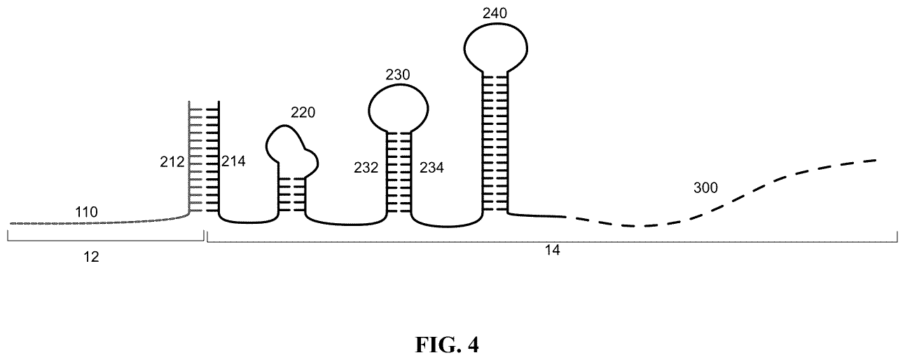

FIGS. 4-9 illustrate several embodiments in which two or more hybridized polynucleotides may form a hybridized guide nucleic acid.

In some embodiments, the first polynucleotide includes the spacer sequence 110 and the first strand of the stem 212 of the tracr region, and the second polynucleotide includes the second strand of the stem 214 to the 3′ end of the tracr region and the additional region 300, which includes an editing template and a PBS. A schematic representation of one of these embodiments is shown in FIG. 4, wherein the first polynucleotide is represented by 12 and the second polynucleotide is represented by 14.

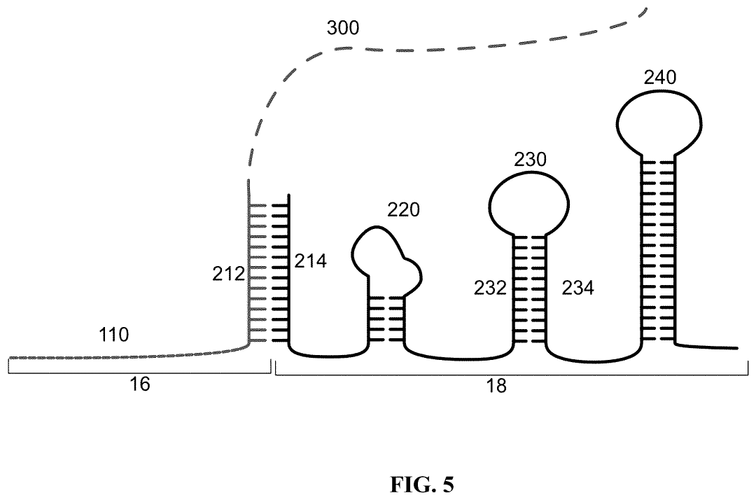

In some embodiments, the first polynucleotide includes the tracr region without the first strand of the stem 212 and the second polynucleotide comprises the spacer sequence 110, the additional region 300, which includes an editing template and a PBS, and the first strand of the stem 212. A schematic representation of one of these embodiments is shown in FIG. 5, wherein the first polynucleotide is represented by 18 and the second polynucleotide is represented by 16.

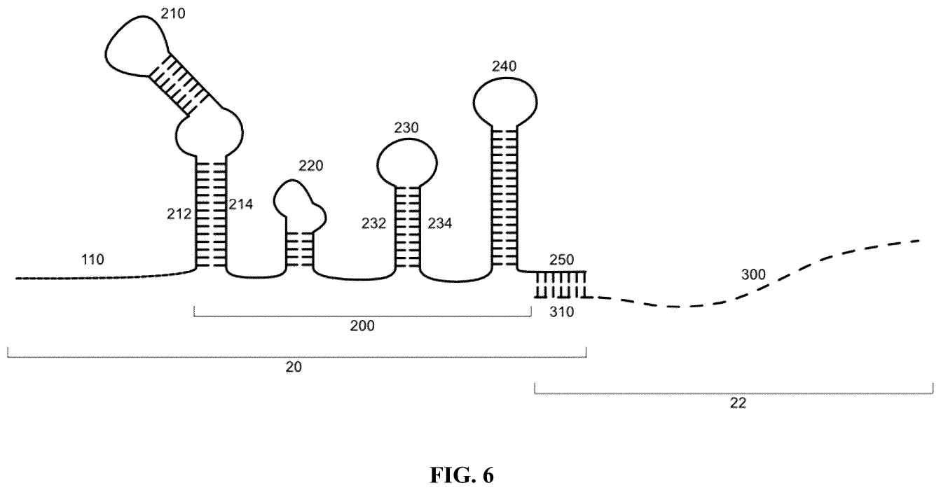

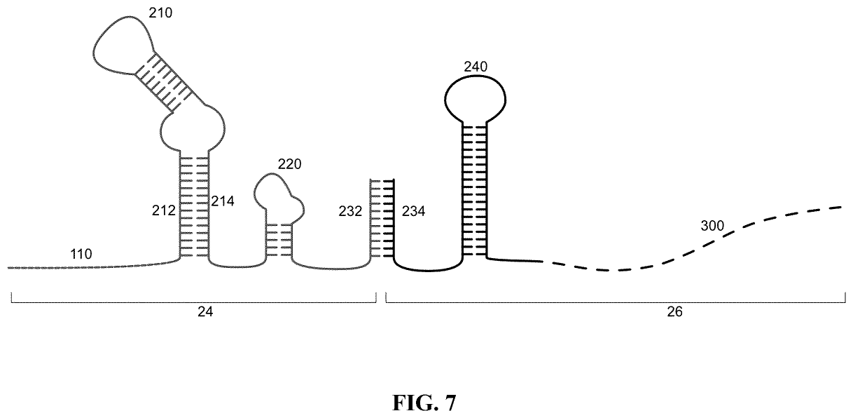

In some embodiments, the first polynucleotide includes the complete tracr region sequence 200, the spacer sequence 110, and a first hybridization sequence on the 3′ end 250, and the second polynucleotide includes the additional region 300, which included the editing template and PBS, and a second hybridization sequence 310. A schematic representation of one of these embodiments is shown in FIG. 6, wherein the first polynucleotide is represented by 20 and the second polynucleotide is represented by 22.

In some embodiments, the first polynucleotide comprises the spacer sequence 110 and the tracr region up to the first hairpin strand 232, and the second polynucleotide comprises the tracr region from the second hairpin strand 234 to the 3′ end of the tracr region and the additional region 300, which includes an editing template and a PBS. A schematic representation of one of these embodiments is shown in FIG. 7, wherein the first polynucleotide is represented by 24 and the second polynucleotide is represented by 26.

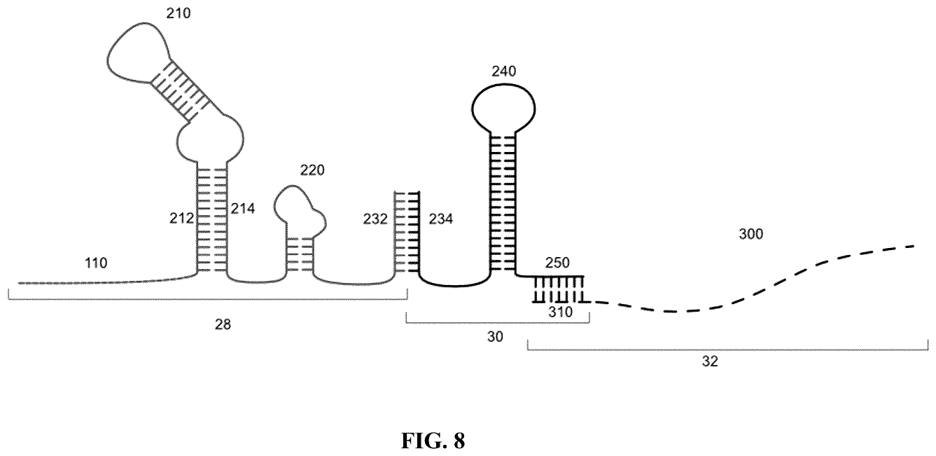

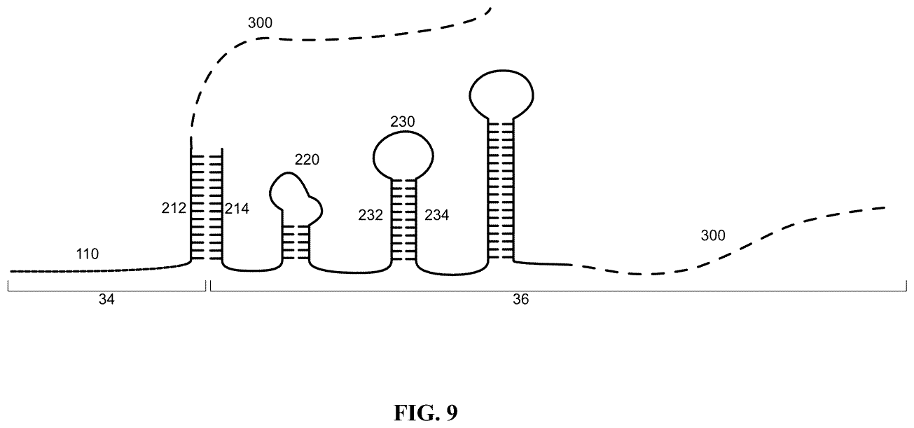

In some embodiments, the first polynucleotide includes the spacer region 110 and the tracr region up to the first hairpin strand 232, the second polynucleotide includes the tracr region from the second hairpin strand 234 to the 3′ end of the tracr region and a hybridization sequence 250, and the third polynucleotide includes the additional region 300, which includes an editing template and a PBS, and a hybridization sequence 310. A schematic representation of one of these embodiments is shown in FIG. 8, wherein the first polynucleotide is represented by 28, the second polynucleotide is represented by 30, and the third polynucleotide is represented by 32.

In some embodiments, the first polynucleotide includes the spacer region 110, the tracr region up to the first hairpin strand 232, and a first additional region 300, including a first editing template and a first PBS, and the second polynucleotide includes the tracr region from the second hairpin strand 234 to the 3′ end of the tracr region and a second additional region 300, which includes a second editing template and a second PBS. In some embodiments, the first editing template and the second editing template may encode the same edit. In some embodiments, the first editing template and the second editing template may have the same sequence. In some embodiments, the first ending template and the second editing template may encode different edits. In some embodiments, the first ending template and the second editing template may have different sequences. A schematic representation of one of these embodiments is shown in FIG. 9, wherein the first polynucleotide is represented by 34 and the second polynucleotide is represented by 36.

### Assembly of Hybridized Guide Nucleic Acid

In another aspect, this disclosure describes methods of producing the hybridized guide nucleic acid described herein. In an embodiment, a method for forming a hybridized guide nucleic acid includes hybridizing a first polynucleotide and a second polynucleotide, wherein hybridizing the first and second polynucleotides includes hybridizing a first hybridization sequence of the first polynucleotide to a second hybridization sequence of the second polynucleotide.

In another embodiment, a method for forming a hybridized guide nucleic acid includes hybridizing a first polynucleotide and a second polynucleotide, wherein hybridizing the first and second polynucleotides includes hybridizing a first hybridization sequence of the first polynucleotide to a second hybridization sequence of the second polynucleotide; and hybridizing the second polynucleotide and a third polynucleotide, wherein hybridizing the second polynucleotide and the third polynucleotide comprises hybridizing a third hybridization sequence of the second polynucleotide to a fourth hybridization sequence of the third polynucleotide.

In another embodiment, a method for forming a hybridized guide nucleic acid includes hybridizing a plurality of polynucleotides. Each polynucleotide comprises at least one hybridization sequence. Hybridizing the plurality of polynucleotides comprises hybridizing a hybridization sequence of each polynucleotide to a hybridization sequence of another polynucleotide.

In some embodiments, hybridizing the hybridization sequences includes annealing the hybridization sequences of denatured polynucleotides. The method may further include a step of initially denaturing the polynucleotides. Denatured polynucleotides may be produced by any suitable method. Denaturing may include increasing temperature and/or increasing the concentration of salt. The temperature may be increased to at least 50° C., at least 60° C., at least ° C., at least 80° C., or at least 90° C. In embodiments, the temperature is increased to no more than 130° C. The concentration of salt may be increased to at least 0.1 M, at least 0.5 M, at least 1.0 M, or at least 5.0 M. In embodiments, the salt is increased to no more than 10 M. The salt may be a simple salt, a basic salt, a neutral salt, or a complex salt. In some embodiments, the salt may be a chaotropic agent such as guanidinium chloride.

In embodiments, annealing the hybridization sequences is accomplished by decreasing temperature. Decreasing temperature may follow a gradient protocol. The temperature may be decreased over a set period of time. The temperature may be decreased over at least 5 minutes, at least 10 minutes, at least 30 minutes, or at least 60 minutes. In embodiments, the temperature is decreased over no more than 180 minutes.

In embodiments, annealing the hybridization sequences is accomplished by decreasing salt concentration. The salt concentration may be decreased by dialysis, dilution, desalting column, or by another method not described herein.

### Pharmaceutical Compositions

In one or more aspects, provided herein is a pharmaceutical composition comprising the hybridized guide nucleic acid or components thereof or the gene editor system as provided herein and a pharmaceutically acceptable carrier or excipient. In one or more aspects, provided herein is a pharmaceutical composition for gene modification comprising a hybridized guide nucleic acid or components thereof as described herein and a gene editor or a nucleic acid sequence encoding the gene editor or components thereof and a pharmaceutically acceptable carrier. Pharmaceutical compositions are formulated in a conventional manner using one or more pharmaceutically acceptable inactive ingredients that facilitate processing of the active compounds into preparations that can be used pharmaceutically. Suitable pharmaceutically acceptable additives are generally well known in the art. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

A pharmaceutical composition can comprise any suitable molar ratio of a hybridized guide nucleic acid or components thereof as described herein or a nucleic acid encoding the hybridized guide nucleic acid or components thereof to gene editor or a nucleic acid sequence encoding the gene editor. In embodiments, the ratio of the hybridized guide nucleic acid or components thereof (or nucleic acid sequence encoding the hybridized guide nucleic acid or components thereof) to the gene editor (or nucleic acid encoding the gene editor) is from 10:1 to 1:10, such as from 5:1 to 1:5, 3:1 to 1:3, or 2:1 to 1:2. In some embodiments, the ratio is 1:1 to 1:3. In some embodiments, the ratio is about 1:2. In some embodiments, the ratio is 1:1.5 to 1:2.5. Thus, for example, the weight ratio of hybridized guide nucleic acid or components thereof to mRNA encoding the gene editor may be 1:1 to 1:3 or 3:1.

A pharmaceutical composition can be a mixture of a hybridized guide nucleic acid or components thereof as described herein or a nucleic acid sequence encoding the hybridized guide nucleic acid or components thereof and a gene editor or a nucleic acid sequence encoding the gene editor with one or more of other chemical components (i.e., pharmaceutically acceptable ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration to an organism or a subject in need thereof.

The pharmaceutical compositions of the present disclosure can be administered to a subject using any suitable methods known in the art. The pharmaceutical compositions described herein can be administered to the subject in a variety of ways, including parenterally, intravenously, intradermally, intramuscularly, colonically, rectally, or intraperitoneally. In one or more embodiments, the pharmaceutical compositions can be administered by intraperitoneal injection, intramuscular injection, subcutaneous injection, or intravenous injection of the subject. In one or more embodiments, the pharmaceutical compositions can be administered parenterally, intravenously, intramuscularly, or orally. In one embodiment, the pharmaceutical composition is comprises a pharmaceutically acceptable solution comprising an LNP encapsulating one or more gRNA and mRNA encoding the editor protein(s) engineered to install a nonsense mutation, splice site mutation, and/or non-synonymous mutation in the LPA gene, as described herein, that is administered intravenously to a subject in need thereof, the LNP may or may not include GalNAc (e.g., a GalNAc-lipid) as described herein. The LNP may be a LNP as described in, for example, U.S. Pat. No. 11,207,416 B2, entitled “Compositions and methods for targeted RNA delivery,” having Verve Therapeutics Inc. as the assignee, and which issued on Dec. 28, 2021.

In one or more embodiments, a pharmaceutical composition for gene modification includes a further therapeutic agent. The additional therapeutic agent may modulate different aspects of the disease, disorder, or condition being treated and provide a greater overall benefit than administration of the therapeutic agent alone. Therapeutic agents include, but are not limited to, a chemotherapeutic agent, a radiotherapeutic agent, a hormonal therapeutic agent, and/or an immunotherapeutic agent. In one or more embodiments, the therapeutic agent may be a radiotherapeutic agent. In one or more embodiments, the therapeutic agent may be a hormonal therapeutic agent. In one or more embodiments, the therapeutic agent may be an immunotherapeutic agent. In one or more embodiments, the therapeutic agent is a chemotherapeutic agent. Preparation and dosing schedules for additional therapeutic agents can be used according to manufacturers' instructions or as determined empirically by a skilled practitioner.

### Kits

It is contemplated herein that the therapeutic agents or drug substances disclosed herein are part of a kit as described herein. Accordingly, one aspect of the disclosure relates to kits including the compositions comprising a hybridized guide nucleic acid or components thereof as provided herein, the gene editor or gene editor system as provided herein, the compositions as provided herein, and/or the lipid nanoparticle formulations as provided herein for treating or preventing a condition. The kits can further include one or more additional therapeutic regimens or agents for treating or preventing a condition.

Also disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material (including use and/or disposal instructions) suitable for a selected formulation and intended mode of administration and treatment.

For example, the container(s) include a composition as described herein, and optionally in addition with therapeutic regimens or agents disclosed herein. Such kits optionally include an identifying description or label or instructions relating to its use in the methods described herein.

A kit typically includes labels listing contents and/or instructions for use, and package inserts with instructions for use. A set of instructions will also typically be included.

In embodiments, a label is on or associated with the container. In one embodiment, a label is on a container when letters, numbers or other characters forming the label are attached, molded, or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

### Dosing

The skilled artisan will appreciate that certain factors may influence the dosage and frequency of administration required to effectively treat a subject, including but not limited to the severity of the disease or disorder, previous treatments, the general characteristics of the subject including health, sex, weight and/or age of the subject, and other diseases present. Moreover, treatment of a subject with a therapeutically effective amount of the compositions can include a single treatment or, preferably, can include a series of treatments. It will also be appreciated that the effective dosage of the composition of the disclosure used for treatment may increase or decrease over the course of a particular treatment. Changes in dosage may result and become apparent from the results of diagnostic assays as described herein. The therapeutically effective dosage will generally be dependent on the patient's status at the time of administration. The precise amount can be determined by routine experimentation but may ultimately lie with the judgment of the clinician, for example, by monitoring the patient for signs of disease and adjusting the treatment accordingly.

Frequency of administration may be determined and adjusted over the course of therapy, and is generally, but not necessarily, based on treatment and/or suppression and/or amelioration and/or delay of a disease. Alternatively, sustained continuous release formulations of a polypeptide or a polynucleotide may be appropriate. Various formulations and devices for achieving sustained release are known in the art. It one embodiment, the pharmaceutical composition comprising the gene editor system for installing a nonsense mutation, splice site mutation, and/or non-synonymous mutation in the LPA gene, as described herein, is dosed once to a subject in need with no need for additional dosing. In another embodiment, the pharmaceutical composition comprising the gene editor system for installing a nonsense mutation, splice site mutation, and/or non-synonymous mutation in the LPA gene, as described herein, is dosed twice with the second dose following sequentially after the first. In another embodiment, the pharmaceutical composition comprising the gene editor system for installing a nonsense mutation, splice site mutation, and/or non-synonymous mutation in the LPA gene, as described herein, is dosed sequentially multiple times until a sufficient amount of therapeutically effective editing in the LPA gene, as described herein, is achieved, with the second dose following sequentially after the first. The progress of this therapy is easily monitored by conventional techniques and assays and may be determined by monitoring blood phenylalanine levels. It should also be understood that the subject may be dosed with LNPs that contain different gene editor system components (e.g., mRNA encoding the gene editor in one LNP and gRNA in another, a mixture of mRNA and gRNA in the same LNP, or different gRNAs in different LNPs with or without mRNA).

The dosing regimen (including a composition disclosed herein) can vary over time. In one or more embodiments, it is contemplated that for an adult subject of normal weight, doses ranging from about 0.01 to 1000 mg/kg may be administered. In one or more embodiments, the dose is between 1 to 200 mg/kg of subject body weight. In one or more embodiments the dosing may be between 0.03 mg/kg to 3 mg/kg, 0.1 to 2 mg/kg; 0.5 to 1.5 mg/kg or anywhere between any of the foregoing ranges. The particular dosage regimen, i.e., dose, timing, and repetition, will depend on the particular subject and that subject's medical history, as well as the properties of the polypeptide or the polynucleotide (such as the half-life of the polypeptide or the polynucleotide, and other considerations well known in the art).

The appropriate therapeutic dosage of a composition as described herein will depend on the specific agent (or compositions thereof) employed, the formulation and route of administration, the type and severity of the disease, whether the polypeptide or the polynucleotide is administered for preventive or therapeutic purposes, previous therapy, the subject's clinical history and response to the antagonist, and the discretion of the attending physician. Typically, the clinician will administer a polypeptide until a dosage is reached that achieves the desired result.

Administration of one or more compositions can be continuous or intermittent, depending, for example, upon the recipient's physiological condition, whether the purpose of the administration is therapeutic or prophylactic, and other factors known to skilled practitioners. The administration of a composition may be essentially continuous over a preselected period of time or may be in a series of spaced dose, e.g., either before, during, or after developing a disease.

The methods and compositions of the disclosure described herein including embodiments thereof can be administered with one or more additional therapeutic regimens or agents or treatments, which can be co-administered to the mammal. By “co-administering” is meant administering one or more additional therapeutic regimens or agents or treatments and the composition of the disclosure sufficiently close in time to enhance the effect of one or more additional therapeutic agents, or vice versa. In this regard, the composition of the disclosure described herein can be administered simultaneously with one or more additional therapeutic regimens or agents or treatments, at a different time, or on an entirely different therapeutic schedule (e.g., the first treatment can be daily, while the additional treatment is weekly). For example, in embodiments, the secondary therapeutic regimens or agents or treatments are administered simultaneously, prior to, or subsequent to the composition of the disclosure.

### OTHER EMBODIMENTS

From the foregoing description, it will be apparent that variations and modifications may be made to the disclosure described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment, any portion of the embodiment, or in combination with any other embodiments or any portion thereof.

As is set forth herein, it will be appreciated that the disclosure comprises specific embodiments and examples of hybridized guide nucleic acids and template-based gene editing systems employing such hybridized guide nucleic acids to effect an edit in a gene and methods of using same for treatment of disease including compositions that comprise such base editing systems, designs and modifications thereto; and specific examples and embodiments describing the synthesis, manufacture, use, and efficacy of the foregoing individually and in combination.

While specific examples and numerous embodiments have been provided to illustrate aspects and combinations of aspects of the foregoing, it should be appreciated and understood that any aspect, or combination thereof, of an exemplary or disclosed embodiment may be excluded therefrom to constitute another embodiment without limitation and that it is contemplated that any such embodiment can constitute a separate and independent claim. Similarly, it should be appreciated and understood that any aspect or combination of aspects of one or more embodiments may also be included or combined with any aspect or combination of aspects of one or more embodiments and that it is contemplated herein that all such combinations thereof fall within the scope of this disclosure and can be presented as separate and independent claims without limitation. Accordingly, it should be appreciated that any feature presented in one claim may be included in another claim; any feature presented in one claim may be removed from the claim to constitute a claim without that feature; and any feature presented in one claim may be combined with any feature in another claim, each of which is contemplated herein. The following enumerated clauses are further illustrative examples of aspects and combination of aspects of the foregoing embodiments and examples:

Following is an example of enumerated clauses:
1. A hybridized guide nucleic acid for use with a template-based editor, comprising:
   a first polynucleotide comprising a first guide nucleic acid element and a first hybridization sequence; and
   a second polynucleotide comprising a second guide nucleic acid element and a second hybridization sequence,
   wherein the second guide nucleic acid element is an editing template,
   wherein the first and second hybridization sequences are complementary, and
   wherein the first and second hybridization sequences are hybridized to one another.
2. The hybridized guide nucleic acid of clause 1, wherein the second polynucleotide further comprises a third hybridization sequence, and wherein the hybridized guide nucleic acid further comprises a third polynucleotide comprising a third guide nucleic acid element and a fourth hybridization sequence,
   wherein the third and fourth hybridization sequences are complementary, and
   wherein the third and fourth hybridization sequences are hybridized to one another.
3. A hybridized guide nucleic acid for use with a template based-editor, the hybridized guide nucleic acid comprising a plurality of polynucleotides,
   wherein each polynucleotide comprises at most 80 nucleotides in length,
   wherein each polynucleotide comprises a gRNA element and a hybridization sequence,
   wherein at least one of the polynucleotides comprises an editing template;
   wherein each hybridization sequence is complementary to at least one other hybridization sequence, and
   wherein the plurality of polynucleotides are hybridized to one another to form the hybridized guide nucleic acid, wherein the hybridized guide nucleic acid comprises a plurality of guide nucleic acid elements.
4. The hybridized guide nucleic acid of any one of clauses 1 to 3, wherein one or more of the polynucleotides comprises more than one guide nucleic acid element.
5. The hybridized guide nucleic acid of any one of clauses 1 to 4, wherein at least one of the guide nucleic acid elements is a spacer region.
6. The hybridized guide nucleic acid of clause 5, wherein the spacer region is 10-60, 15-40, or 20 nucleotides in length.
7 The hybridized guide nucleic acid of clause 5 or 6, wherein the spacer region is complementary to a region of the genome.
8. The hybridized guide nucleic acid of any one of clauses 1 to 7, wherein at least one of the guide nucleic acid elements comprises a tracr region or a portion thereof.
9. The hybridized guide nucleic acid of clause 8, wherein the tracr region comprises a stem comprising a first strand and a second strand and a hairpin comprising a first strand and a second strand.
10. The hybridized guide nucleic acid of clause 8 or 9, wherein the guide nucleic acid element comprising the tracr region or the portion thereof comprises the complete tracr region.
11. The hybridized guide nucleic acid of clause 8 or 9, wherein the guide nucleic acid element comprising the tracr region or the portion thereof comprises a portion of the tracr region, and wherein the portion of the tracr region comprises the first strand of the stem.
12. The hybridized guide nucleic acid of clause 8 or 9, wherein the guide nucleic acid element comprising the tracr region or the portion thereof comprises a portion of the tracr region, and wherein the portion of the tracr region comprises the second strand of the stem.
13. The hybridized guide nucleic acid of clause 8 or 9, wherein the guide nucleic acid element comprising the tracr region or the portion thereof comprises a portion of the tracr region, and wherein the portion of the tracr region comprises the first strand of the hairpin.
14. The hybridized guide nucleic acid of clause 8 or 9, wherein the guide nucleic acid element comprising the tracr region or the portion thereof comprises a portion of the tracr region, and wherein the portion of the tracr region comprises the second strand of the hairpin.
15. The hybridized guide nucleic acid of any one of clauses 8 to 14, wherein at least one of the hybridization sequences is in the tracr region.
16. The hybridized guide nucleic acid of clause 15, wherein the hybridization sequence comprises at least a portion of strand of a stem-loop or of a hairpin of the tracr region.
17. The hybridized guide nucleic acid of any one of clauses 1 to 16, at least one of the polynucleotides comprises a primer binding site.
18. The hybridized guide nucleic acid of clause 17, wherein the primer binding site is homologous to region of a gene
19. The hybridized guide nucleic acid of any one of clauses 1 to 18, wherein the editing template is 10 to 60 nucleotides in length.
20. The hybridized guide nucleic acid of any one of clauses 1 to 19, wherein at least one of the guide nucleic acid elements is an RNA stabilizing motif.
21. The hybridized guide nucleic acid of clause 20, wherein the RNA stabilizing motif is F6, PP7, or MS2
22. The hybridized guide nucleic acid of any one of clauses 1 to 21, wherein at least one of hybridization sequences comprise 10-30 nucleotides.
23. The hybridized guide nucleic acid of any one of clauses 1 to 22, wherein at least one of the hybridization sequences is on a 3′ end of at least one of the polynucleotides.
24. The hybridized guide nucleic acid of any one of clauses 1 to 23, wherein at least one of the hybridization sequences is on a 5′ end of at least one of the polynucleotides.
25. The hybridized guide nucleic acid of any one of clauses 1 to 24, wherein at least one of the hybridization sequences is not on either end of at least one of the polynucleotides.
26. The hybridized guide nucleic acid of any one of clauses 1 to 25, wherein one or more of the polynucleotides is chemically modified.
27. The hybridized guide nucleic acid of clause 26, wherein the chemically modified polynucleotide comprises a 2′-O-methyl modification, a phosphothioate modification, or both.
28. The hybridized guide nucleic acid of any one of clauses 1 to 27, wherein the polynucleotides comprise DNA nucleotides, RNA nucleotides, or a combination thereof.
29. The hybridized guide nucleic acid of any of clauses 1 to 28, wherein one of the polynucleotides comprises a spacer region and a first strand of a stem of a tracr region, and another of the polynucleotides comprises a tracr region without the strand of the stem.
30. The hybridized guide nucleic acid of any of clauses 1 to 28, wherein one of the polynucleotides comprises a tracr region without a first strand of a stem, and another of the polynucleotides comprises a spacer region and the first strand of the stem of the tracr region.
31. The hybridized guide nucleic acid of any of clauses 1 to 28, wherein one of the polynucleotides comprises a spacer region and a tracr region.
32. The hybridized guide nucleic acid of any of clauses 1 to 28, wherein one of the polynucleotides comprises a spacer region and a tracr region up to a first hairpin strand, and another of the polynucleotides comprises the tracr region from a second hairpin strand to the 3′ end of the tracr region.
33. The hybridized guide nucleic acid of any of clauses 1 to 28, wherein one of the polynucleotides comprises a spacer region and a tracr region up to the first hairpin strand, another of the polynucleotides comprises the tracr region from a second hairpin strand to the 3′ end of the tracr region, and wherein the hybridized guide nucleic acid comprises a third polynucleotide comprises the editing template.
34. The hybridized guide nucleic acid of any of clauses 1 to 28, wherein one of the polynucleotides comprises a spacer region, a tracr region up to a first hairpin strand, and a first editing template, and another of the polynucleotides comprises a tracr region from a second hairpin to the 3′ end of the tracr region and a second editing template.
35. The hybridized guide nucleic acid of clause 34, wherein the sequence of the first editing template and sequence of the second editing template are at least 75%, at least 90%, or 100% identical.
36. The hybridized guide nucleic acid of clause 34, wherein the sequence of the first editing template and the sequence of the second editing template are not substantially similar.
37. A method for forming the hybridized guide nucleic acid of clauses 1 to 36, the method comprising hybridizing each of the polynucleotides to another of the polynucleotides, such that a hybridization sequence of each of the polynucleotides hybridizes a hybridization sequence of another polynucleotide.
38. The method of clause 37, further comprising denaturing the polynucleotides, and wherein hybridizing the polynucleotides comprises annealing the denatured polynucleotides.
39. The method of clause 38, wherein the denaturing comprises increasing temperature.
40. The method of clause 39, wherein the temperature is increased to 70° C. to 200° C., 85° C. to 150° C., or 95° C. to 100° C.
41. The method of any one of clauses 38 to 40, wherein the denaturing comprises increasing salt concentration.
42. The method of clause 41, wherein the salt is a chaotropic agent.
43. The method of any one of clauses 38 to 42, where the annealing comprises decreasing temperature.
44. The method of clause 43, wherein the decreasing temperature follows a gradient protocol.
45. The method of any one of clause 38 to 44, wherein the annealing comprises decreasing salt concentration.
46. The method of clause 45, wherein the salt is a chaotropic agent.
47. A composition comprising:
   a first polynucleotide comprising a first guide nucleic acid element and a first hybridization sequence; and
   a second polynucleotide comprising a second guide nucleic acid element and a second hybridization sequence,
   wherein the second guide nucleic acid element is an editing template,
   wherein the first and second hybridization sequences are complementary, and
   wherein the first and second hybridization sequences are engineered to hybridize to one another to form at least a portion of a functional guide nucleic acid.
48. The composition of clause 47, wherein the second polynucleotide further comprises a third hybridization sequence, and wherein the hybridized guide nucleic acid further comprises a third polynucleotide comprising a third guide nucleic acid element and a fourth hybridization sequence,
   wherein the third and fourth hybridization sequences are complementary, and
   wherein the third and fourth hybridization sequences are engineered to hybridize to one another to hybridize to one another to form at least a portion of a functional guide nucleic acid.
49. A composition comprising:
   a plurality of polynucleotides,
   wherein each polynucleotide comprises at most 80 nucleotides in length,
   wherein each polynucleotide comprises a gRNA element and a hybridization sequence,
   wherein at least one of the polynucleotides comprises an editing template;
   wherein each hybridization sequence is complementary to at least one other hybridization sequence, and
   wherein the plurality of polynucleotides are engineered to hybridize to one another to form a functional guide nucleic acid for use in a template-based guide nucleic acid system.
50. The composition of any one of clauses 47 to 50, wherein one or more of the polynucleotides comprises more than one guide nucleic acid element.
51. The composition of any one of clauses 47 to 51, wherein at least one of the guide nucleic acid elements is a spacer region.
52. The composition of clause 51, wherein the spacer region is 10-60, 15-40, or 20 nucleotides in length.
53. The composition of clause 51 or 52, wherein the spacer region is complementary to a region of the genome.
54. The composition of any one of clauses 47 to 53, wherein at least one of the guide nucleic acid elements comprises a tracr region or a portion thereof.
55. The composition of clause 54, wherein the tracr region comprises a stem comprising a first strand and a second strand and a hairpin comprising a first strand and a second strand.
56. The composition of clause 54 or 55, wherein the guide nucleic acid element comprising the tracr region or the portion thereof comprises the complete tracr region.
57. The composition of clause 54 or 55, wherein the guide nucleic acid element comprising the tracr region or the portion thereof comprises a portion of the tracr region, and wherein the portion of the tracr region comprises the first strand of the stem.
58. The composition of clause 54 or 55, wherein the guide nucleic acid element comprising the tracr region or the portion thereof comprises a portion of the tracr region, and wherein the portion of the tracr region comprises the second strand of the stem.
59. The composition of clause 54 or 55, wherein the guide nucleic acid element comprising the tracr region or the portion thereof comprises a portion of the tracr region, and wherein the portion of the tracr region comprises the first strand of the hairpin.
60. The composition of clause 54 or 55, wherein the guide nucleic acid element comprising the tracr region or the portion thereof comprises a portion of the tracr region, and wherein the portion of the tracr region comprises the second strand of the hairpin.
61. The composition of any one of clauses 54 to 60, wherein at least one of the hybridization sequences is in the tracr region.
62. The composition of clause 61, wherein the hybridization sequence comprises at least a portion of strand of a stem-loop or of a hairpin of the tracr region.
63. The composition of any one of clauses 47 to 62, wherein at least one of the polynucleotides comprises a primer binding site.
64. The composition of clause 63, wherein the primer binding site is homologous to region of a gene
65. The composition of any one of clauses 47 to 64, wherein the editing template is 10 to 60 nucleotides in length.
66. The composition of any one of clauses 47 to 65, wherein at least one of the guide nucleic acid elements is an RNA stabilizing motif.
67. The composition of clause 66, wherein the RNA stabilizing motif is F6, PP7, or MS2
68. The composition of any one of clauses 47 to 67, wherein at least one of hybridization sequences comprise 10-30 nucleotides.
69. The composition of any one of clauses 47 to 68, wherein at least one of the hybridization sequences is on a 3′ end of at least one of the polynucleotides.
70. The composition of any one of clauses 47 to 69, wherein at least one of the hybridization sequences is on a 5′ end of at least one of the polynucleotides.
71. The composition of any one of clauses 47 to 70, wherein at least one of the hybridization sequences is not on either end of at least one of the polynucleotides.
72. The composition of any one of clauses 47 to 71, wherein one or more of the polynucleotides is chemically modified.
73. The composition of clause 72, wherein the chemically modified polynucleotide comprises a 2′-O-methyl modification, a phosphothioate modification, or both.
74. The composition of any one of clauses 47 to 73, wherein the polynucleotides comprise DNA nucleotides, RNA nucleotides, or a combination thereof.
75. The composition of any of clauses 47 to 74, wherein one of the polynucleotides comprises a spacer region and a first strand of a stem of a tracr region, and another of the polynucleotides comprises a tracr region without the strand of the stem.
76. The composition of any of clauses 47 to 74, wherein one of the polynucleotides comprises a tracr region without a first strand of a stem, and another of the polynucleotides comprises a spacer region and the first strand of the stem of the tracr region.
77. The composition of any of clauses 47 to 74, wherein one of the polynucleotides comprises a spacer region and a tracr region.
78. The composition of any of clauses 47 to 74, wherein one of the polynucleotides comprises a spacer region and a tracr region up to a first hairpin strand, and another of the polynucleotides comprises the tracr region from a second hairpin strand to the 3′ end of the tracr region.
79. The composition of any of clauses 47 to 28, wherein one of the polynucleotides comprises a spacer region and a tracr region up to the first hairpin strand, another of the polynucleotides comprises the tracr region from a second hairpin strand to the 3′ end of the tracr region, and wherein the hybridized guide nucleic acid comprises a third polynucleotide comprises the editing template.
80. The composition of any of clauses 1 to 28, wherein one of the polynucleotides comprises a spacer region, a tracr region up to a first hairpin strand, and a first editing template, and another of the polynucleotides comprises a tracr region from a second hairpin to the 3′ end of the tracr region and a second editing template.
81. The composition of clause 80, wherein the sequence of the first editing template and sequence of the second editing template are at least 75%, at least 90%, or 100% identical.
82. The composition of clause 80, wherein the sequence of the first editing template and the sequence of the second editing template are not substantially similar.
83. A template-based gene editor system comprising:
   one or more mRNAs encoding a template-based gene editor or components thereof;
   a hybridized guide according to any one of clauses 1 to 36, or components thereof.
84. A pharmaceutical composition comprising the template-based editor system according to clause 83.
85. The pharmaceutical composition of clause 84, further comprising a lipid nanoparticle (LNP).
86. The pharmaceutical composition of clause 85, wherein the lipid nanoparticle comprises the one or more mRNAs encoding a template-based gene editor or components thereof and comprises the hybridized guide or components thereof.

It will also be appreciated from reviewing the present disclosure, that it is contemplated that the one or more aspects or features presented in one of or a group of related clauses may also be included in other clauses or in combination with the one or more aspects or features in other clauses.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

### EXAMPLES

The present disclosure is illustrated by the following examples. It is to be understood that the particular examples, materials, amounts, and procedures are to be interpreted broadly in accordance with the scope and spirit of the disclosure as set forth herein.

### Example 1. Synthesis of Hybridized Guide Nucleic Acids

Guide nucleic acids were synthesized using standard solid phase oligonucleotide synthesis methods with an automated synthesizer. Examples of automated synthesizers include K&A H-8 SE DNA/RNA synthesizer (available from Sierra BioSystems™, Sonora, CA), MERMADE 6, 12, 48, or 96 synthesizers (available from Biosearch Technologies™, Petaluma, CA); ÄKTA OLIGOPILOT (available from Cytiva™, Marlborough, MA); DR OLIGO (available from Biolytic Lab Performance Inc.™, Fremont, CA); ABI synthesizer (Biolytic Lab Performance Inc.™); and EXPEDITE DNA/RNA synthesizer (available from Applied Biosystems™, Waltham, MA).

Guide nucleic acids were grown on a 500-600 angstrom (Å), 1000 Å, 1500 Å, 2000 Å, 2500 Å or a 3000 Å standard controlled pore glass support with loading capacity ranging from 20 μmol/g to 120 μmol/g or on a polymer support with loading capacity ranging from 20 μmol/g to 250 μmol/g by stepwise incorporation of commercially available nucleotide phosphoramidite starting materials/building blocks and/or modified nucleotide phosphoramidite starting materials/building blocks. Nucleotide phosphoramidite starting materials included a phosphite conjugated to the 3′ O of the sugar. The phosphite included a protecting group (e.g., 2-cyanoethyl group) and a reactive group (e.g., diisopropylamino group). Nucleotide and modified nucleotide phosphoramidite starting materials included a nucleoside component. The ribosugar component of nucleoside containing phosphoramidite starting materials included a 5′-OH protecting group (e.g., dimethoxytrityl (DMTr), or Monomethoxytritly (MMTr)) and a 2′-OH protecting group (e.g., tert-butyldimethylsilyl group). Examples of the nucleobase components of the phosphoramidite starting materials include uridine, 4-N-acetylcytidine; 6-N-benzoyladenosine; and 2-N-isobutyrylguanosine or other exocyclic amine protection used in the industry for manufacturing oligonucleotides under solid phase synthesis conditions. For the incorporation of 2′-O-methylribonucleotides, the nucleoside component of the phosphoramidite precursor included the 2′-O-methyl group. Based on the oxidizing reagent used, the phosphoramidite precursors were used to form phosphate (PO) or phosphorothioate (PS) backbone linkages. Protected phosphoramidite precursors (e.g., building blocks) enable synthesis of guide nucleic acids through the repetition of coupling, oxidation, capping, and deprotection steps.

Example precursors and resulting residues are shown in Table 1.

TABLE 1
Examples of precursors and resulting residues

| Phosphoramidite Precursor | Resulting Incorporated Residue |
|---|---|
| 5′-O-(DMTr)-2′-O-methylribo-3′-O-(2- cyanoethyl-N,N-diisopropyl)phos- phoramidite | 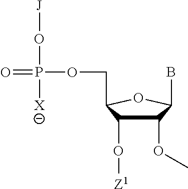 |
| | where B is a nucleobase |
| 5′-O-(4,4′-dimethoxytrityl)-2′-O-(tert- butyldimethylsilyl)ribo-3′-O-(2-cyanoethyl- N,N-diisopropyl)phosphoramidite | 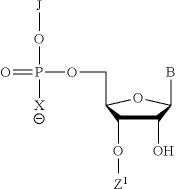 |
| | where B is a nucleobase |

“X” is sulfur (S) when the oxidizing reagent is [(9 dimethylaminomethylene)amino]-3H-1,2,4-dithiazole-5-thione. “X” is oxygen (O) when the oxidizing reagent is I₂/H₂O for prepapring the phosphorothioate backbone and phosphodiester (phosphate) backbone respectively. “Q” is oxygen or sulfur.

In the first step, the phosphite of an activated phosphoramidite precursor was coupled to the 5′ OH of a nucleotide immobilized on a controlled pore glass or on a polymer support. Phosphoramidites were activated using 5-(benzylthio)-1H-tetrazole (BTT), 5-(ethylthio)-1H-tetrazole (ETT) or 4,5-dicyanoimidazole (DCI) and were reacted with to the 5′ OH of the solid bound nucleotide at room temperature for between two minutes and 30 mins under solid phase synthesis conditions. For the preparation of a guide nucleic acid with non-nucleotide moiety at the 3′-end, the activated phosphoramidite was coupled to a hydroxyl group of the non-nucleotide moiety immobilized on a controlled pore glass or on a polymer support.

In the second step, capping or oxidation was performed. Whether capping or oxidation was performed depended on the requirement of phosphate or phosphorothioate linkages. An oxidizing reagent was used to oxidize the phosphite to a phosphate or phosphorothioate group. An optional capping step was used to cap the unreacted 5′ termini (5′ termini that did not undergo a coupling reaction with the activated phosphoramidite during coupling (i.e., the first step)). During the introduction of phosphorothioate linkage, oxidation was performed before capping. To form a phosphorothioate backbone linkage, the oxidizing reagent was a sulfur transfer reagent (e.g., [(9 dimethylaminomethylene)amino]-3H-1,2,4-dithiazole-5-thione (DDTT)). During the oxidizing reaction, a 0.05 M or 0.1 M solution of the sulfur transfer reagent DDDT in pyridine was administered to the reaction column containing the controlled pore glass support or the polymer support. The reaction proceeded at room temperature (e.g., 20° C.-25° C.) for three minutes to 20 mins.

The oxidizing reagent used for phosphate formation did not include sulfur (e.g., elemental iodine and water in pyridine or lutidine). An example of oxidizing reaction solution for the formation of a phosphate was 0.05 M I₂ in pyridine-H₂O (90% pyridine, 10% water (v/v)) and the reaction takes place at room temperature in 1-10 min. A capping step was performed before and after oxidation, during the formation of phosphate linkages. Cap mix A (N-methylimidazole:2,6-lutidine:acetonitrile 2:3:5) and cap mix B (Acetic anhydride 20-40% (v/v) in acetonitrile) were used for capping in both instances, irrespective of phosphate or phosphorothioate linkage formation. Capping reaction were allowed to proceed for 30 seconds or 3 minutes to 6 minutes at room temperature.

In a fourth step, the DMTr or MMTr group at the 5′ position was removed revealing a free OH for coupling of additional activated phosphoramidite to the growing oligonucleotide chain on the controlled pore glass or polymer support. The oligonucleotide containing controlled pore glass supports were treated with 3% dichloroacetic acid or 3% trichloroacetic acid in dichloromethane or toluene for 1-10 mins at room temperature to remove the DMTr or MMTr protecting group from the 5′-hydroxyl group or the primary alcohol for the incorporation of abasic non-sugar residues.

Steps 1 through 4 were repeated until the synthesis of the oligonucleotides was complete. After completion of the synthesis, a global removal of nucleobase protecting groups (e.g., isobutyryl, acetyl, benzoyl groups, if present) was accomplished using freshly prepared ammonia-methylamine solution (AMA solution; 28% aqueous NH₄OH and 40% water:methylamine 1:1 (v/v)) at 65° C. for 15 mins. The AMA solution was removed and the guide nucleic acids are treated with a cocktail of dimethyl sulfoxide (DMSO) and triethylamine trihydrofluoride (TEA·3HF) (1.5:1 or 1:1) to globally remove the 2′ tert-butyldimethylsilyl (TBDMS) protecting groups.

Following the deprotection steps, oligonucleotides were precipitated using a solution of 3 M sodium acetate (NaOAc) in 2-propanol followed by desalting with 10 kDa cutoff desalting column (e.g., Amicon filters available from Millipore Sigma™, St. Louis, MO). The integrity of the resulting guide nucleic acids was determined by liquid chromatography mass spectrometry (LC-MS) using a BioAccord™ Watersu™ instrument (available from Waters Corporation™, Milford, MA). After confirming the presence of the mass of the desired guide species, the crude products were purified by ion pairing-ion-exchange HPLC (IPAXHPLC). Buffer A (0.1 M TEAA pH 7.0, 7% acetonitrile) and buffer B (0.1 M TEAA pH 7.0, 7% acetonitrile, 1 M NaBr) are used as the mobile phases on a TSKgel SuperQ-5PW (20) column at 70° C. (Tosoh Bioscience™, King of Prussia, PA, US). Purification of crude mixture was achieved by running 0-25% buffer B over 1 column volume (CV), followed by an increase to 25-60% buffer B over 15 CV, followed by 60% to 100% buffer B over 1 CV. The purified fractions were analyzed by mass spectrometry. Fractions with targeted molecular weight fare collected and combined.

Following purification, fractions were lyophilized under vacuum, resuspended in RNase free water and desalted using the same methods as described above. The desalted samples were directly used for further experiments.

### Example 2. Evaluation of RT-Editing of LMNA Gene Using Hybridized Guide

The ability of hybridized guide nucleic acids to effect editing of the human LAMA gene was evaluated. Hybridized guide nucleic acids having a structure similar to that shown in FIG. 7 were generated and tested for ability to introduce an edit in the LMNA gene. The hybridized guides were formed from a first polynucleotide having a 3′ hybridization sequence corresponding generally to strand 232 of the stem shown in FIG. 7 and a second polynucleotide having a 5′ hybridization sequence generally corresponding to strand 234 of the stem shown in FIG. 7. The first polynucleotide (e.g., first polynucleotide 24 shown in FIG. 7) included a spacer sequence (e.g., spacer sequence 110 shown in FIG. 7) and a portion of the tracr region (e.g., to the 3′ end of strand 232 of stem). The second polynucleotide (e.g., second polynucleotide 26 shown in FIG. 7) included an additional region containing an editing template and a PBS (e.g., additional region 300 shown in FIG. 7) and a portion of the tracr region (e.g., to the 5′ end of strand 234 of stem). Among other variables, the length of hybridization sequences (e.g., strands 232, 234 of stem shown in FIG. 7) was varied to determine the effect on editing efficiency. Hybridized guides were generated from the pairs of first and second polynucleotides shown below in Table 3.

The spacer, PBS, and editing templates were designed to introduce an S71I edit (via mutation of G to a T) into the human LAMA gene.

Briefly, editing efficiency was assessed by co-transfection of the hybridized guides with mRNA(s) encoding Cas9-based RT editing proteins and nicking gRNA into Huh7 cells. Editing efficiency at the LMNA S71I locus was read out via a digital PCR (dPCR) assay designed to detect installation of the intended single nucleotide change in the hLMNA gene.

Preparation of the RNA for Transfection (on the Day of Transfection)

For the hybridized gRNA configurations, in which the guide RNA comprises two RNA segments (referred to as “Polynucleotide 1” and “Polynucleotide 2” in this example), gRNA Polynucleotide 1 and gRNA Polynucleotide 2 were combined in a 1:1 mass ratio in a 10 mM Tris, pH 7.5, 0.1 mM EDTA buffer. In some instances, a heat-and-cool procedure was applied at this stage to anneal the two pieces together (data not shown). In some instances, no heat-and-cool procedure was applied. This mixture was then diluted in Opti-MEM reduced serum media. This mixture constitutes Component “A2.” A separate mixture was made containing a single guide RNA designed to install a separate nick and one of two mRNA configurations. For the first mRNA configuration, two mRNAs were used, encoding (a) a Cas9 nickase protein (H840A) and (b) a reverse transcriptase variant fused to an MS2 bacteriophage coat protein (MCP). For the second mRNA configuration, one mRNA encoding the PEmax protein is used. The mRNA(s) of Configuration 1 or 2 were diluted in Opti-MEM reduced serum medium. This mixture constituted Component “A1.” Components A1 and A2 were mixed in a 1:1 volumetric ratio to form Component “A.” Component “B” was made by diluting the Messenger Max transfection reagent in Opti-MEM. Component “A” and component “B” were combined in a 1:1 volumetric ratio to reconstitute the highest dose used in the study. At this stage, the mixture was incubated for at least 5 minutes at room temperature to promote complexation. Any lower doses were created by diluting this transfection mix.

Transfection of Huh7 Cells

One day prior to the transfection, Huh7 cells were counted and assessed for viability. Cells were then seeded into 96 well plates at an approximate seeding density of 15,000 cells per well. On the day of the transfection, media was aspirated, and fresh growth media was added to the wells. Next, the prepared transfection mix was added to the wells and gently swirled to mix. Table 2 shows an example of the final mass fractions and mass concentrations for mRNA Configuration 1 at 315 ng/mL total RNA dose in the cell media. Huh7 cells were incubated with the transfection mixture for 72 hours to allow ample time for editing to occur.

TABLE 2

| | | Final | Final | |
|---|---|---|---|---|
| | | Fraction of | Concentration in | |
| | | Total RNA Mass | Transfection Media | |
| Component | Description | Transfected | for 315 ng/mL Dose | |
| mRNA 1 | mRNA encoding a protein | 0.25 | 78.75 | ng/mL |
| | including a Cas9(H840A) | | | |
| | nickase | | | |
| mRNA 2 | mRNA encoding a protein | 0.25 | 78.75 | ng/mL |
| | including a reverse | | | |
| | transcriptase (RT) enzyme | | | |
| | variant fused to the MS2 | | | |
| | bacteriophage coat protein | | | |
| | (MCP) | | | |
| Split gRNA | gRNA piece including the | 0.167 | 52.5 | ng/mL |
| Piece 1 | spacer, repeat: antirepeat | | | |
| | helix, stem loop 1, and part | | | |
| | of stem loop 2 | | | |
| Split gRNA | gRNA piece including part | 0.167 | 52.5 | ng/mL |
| Piece 2 | of stem loop 2, stem loop 3, | | | |
| | and the template region | | | |
| Nicking | ngRNA | 0.167 | 52 | ng/mL |

gRNA

Lysis

72 hours after transfection, media was aspirated and cells were lysed by adding 50 μL of QuickExtract™ DNA Extraction solution. This was incubated at room temperature for five minutes, followed by pipetting and/or scraping to dislodge cells before transferring to a 96-well PCR plate. A sequence of vortex and heat incubation is used to fully lyse the cells, followed by a spin down to pellet cell debris. 10 μL of this lysate (avoiding the debris pellet) is then transferred to another plate and diluted three-fold in water for use in the subsequent digital PCR assay.

Readout

Digital PCR (dPCR) was performed using the 3× diluted genomic DNA input from the QuickExtract™ process. dPCR was performed according to the manufacturer's instructions with a probe and primer set previously validated by comparing results to a next generation sequencing (NGS) readout (data not shown).

Percent editing was calculated by dividing the number of partitions detected by the edited probe by the sum of the partitions detected by the edited and wild type probes. This fraction is then multiplied by 100% to convert to a percentage.

TABLE 3
Polynucleotide pairs used to produce hybridized guides

| Hybridized | | |
|---|---|---|
| guide no. | Polynucleotide 1 | Polynucleotide 2 |
| 1 | c*c*u*UGAUGCCGGACACCUCGg | ccgugGcaccgagucggugcGAGGUG |
| | UUUUAGagcuaGaaauagcaaGUUaAa | GUCAUCCGCGAGGUGUCCGG |
| | AuAaggcuaGUccGUUAacAAcgg | CA*u*c*a |
| 2 | c*c*u*UGAUGCCGGACACCUCGg | ccgugGcaccgagucggugcGAGGUG |
| | UUUUAGagcuaGaaauagcaaGUUaAa | GUCAUCCGCGAGGUGUCCGG |
| | AuAaggcuaGUccGUUAucAAcgg | CA*u*c*a |
| 3 | c*c*u*UGAUGCCGGACACCUCGg | aagugGcaccgagucggugcGAGGUG |
| | UUUUAGagcuaGaaauagcaaGUUaAa | GUCAUCCGCGAGGUGUCCGG |
| | AuAaggcuaGUccGUUAucAAcuu | CA*u*c*a |
| 4 | c*c*u*UGAUGCCGGACACCUCGg | guccgugGcaccgagucggugcGAGGU |
| | UUUUAGagcuaGaaauagcaaGUUaAa | GGUCAUCCGCGAGGUGUCCG |
| | AuAaggcuaGUccGUUAacAAcggac | GCA*u*c*a |
| 5 | c*c*u*UGAUGCCGGACACCUCGg | aaaagugGcaccgagucggugcGAGGU |
| | UUUUAGagcuaGaaauagcaaGUUaAa | GGUCAUCCGCGAGGUGUCCG |
| | AuAaggcuaGUccGUUAucAAcuuuu | GCA*u*c*a |
| 6 | c*c*u*UGAUGCCGGACACCUCGg | ccccgugGcaccgagucggugcGAGGU |
| | UUUUAGagcuaGaaauagcaaGUUaAa | GGUCAUCCGCGAGGUGUCCG |
| | AuAaggcuaGUccGUUAucAAcgggg | GCA*u*c*a |
| 7 | c*c*u*UGAUGCCGGACACCUCGg | ggccgugGcaccgagucggugcGAGGU |
| | UUUUAGagcuaGaaauagcaaGUUaAa | GGUCAUCCGCGAGGUGUCCG |
| | AuAaggcuaGUccGUUAucAAcggcc | GCA*u*c*a |
| 8 | c*c*u*UGAUGCCGGACACCUCGg | aaccgugGcaccgagucggugcGAGGU |
| | UUUUAGagcuaGaaauagcaaGUUaAa | GGUCAUCCGCGAGGUGUCCG |
| | AuAaggcuaGUccGUUAucAAcgguu | GCA*u*c*a |
| 9 | c*c*u*UGAUGCCGGACACCUCGg | ccgugugGcaccgagucggugcGAGGU |
| | UUUUAGagcuaGaaauagcaaGUUaAa | GGUCAUCCGCGAGGUGUCCG |
| | AuAaggcuaGUccGUUAacAAcacgg | GCA*u*c*a |
| 10 | c*c*u*UGAUGCCGGACACCUCGg | guguaagugGcaccgagucggugcGAG |
| | UUUUAGagcuaGaaauagcaaGUUaAa | GUGGUCAUCCGCGAGGUGUC |
| | AuAaggcuaGUccGUUAucAAcuuaca | CGGCA*u*c*a |
| | c | |
| 11 | c*c*u*UGAUGCCGGACACCUCGg | guccaagugGcaccgagucggugcGAG |
| | UUUUAGagcuaGaaauagcaaGUUaAa | GUGGUCAUCCGCGAGGUGUC |
| | AuAaggcuaGUccGUUAucAAcuugga | CGGCA*u*c*a |
| | c | |
| 12 | c*c*u*UGAUGCCGGACACCUCGg | g*c*a*CAUGAGGAUCACCCAU |
| | UUUUAGagcuaGaaauagcaaGUUaAa | GUGCuuGCACAUGAGGAUCAC |
| | AuAaggcuaGUccGUUAucAAcuugga | CCAUGUGCuuguccaagugGcaccga |
| | c | gucggugcGAGGUGGUCAUCCG |
| | | CGAGGUGUCCGGCA*u*c*a |
| 13 | c*c*u*UGAUGCCGGACACCUCGg | g*c*a*CAUGAGGAUCACCCAU |
| | UUUUAGagcuaGaaauagcaaGUUaAa | GUGCuuGCACAUGAGGAUCAC |
| | AuAaggcuaGUccGUUAucAAcuugga | CCAUGUGCguccaagugGcaccgagu |
| | c | cggugcGAGGUGGUCAUCCGCG |
| | | AGGUGUCCGGCA*u*c*a |
| 14 | c*c*u*UGAUGCCGGACACCUCGg | g*c*a*CAUGAGGAUCACCCAU |
| | UUUUAGagcuaGaaauagcaaGUUaAa | GUGCuuguccaagugGcaccgagucgg |
| | AuAaggcuaGUccGUUAucAAcuugga | ugcGAGGUGGUCAUCCGCGAG |
| | c | GUGUCCGGCA*u*c*a |
| 15 | c*c*u*UGAUGCCGGACACCUCGg | g*c*a*CAUGAGGAUCACCCAU |
| | UUUUAGagcuaGaaauagcaaGUUaAa | GUGCguccaagugGcaccgagucggugc |
| | AuAaggcuaGUccGUUAucAAcuugga | GAGGUGGUCAUCCGCGAGGU |
| | c | GUCCGGCA*u*c*a |
| 16 | c*c*u*UGAUGCCGGACACCUCGg | guccaagugGcaccgagucggugcGAG |
| | UUUUAGagcuaGaaauagcaaGUUaAa | GUGGUCAUCCGCGAGGUGUC |
| | AuAaggcuaGUccGUUAucAAcuugga | CGGCA*u*c*a |
| | cuuGCACAUGAGGAUCACCCAUG | |
| | *u*g*c | |
| 17 | c*c*u*UGAUGCCGGACACCUCGg | guccaagugGcaccgagucggugcGAG |
| | UUUUAGagcuaGaaauagcaaGUUaAa | GUGGUCAUCCGCGAGGUGUC |
| | AuAaggcuaGUccGUUAucAAcuugga | CGGCA*u*c*a |
| | cGCACAUGAGGAUCACCCAUG*u | |
| | *g*c | |
| 18 | c*c*u*UGAUGCCGGACACCUCGg | guccguccaagugGcaccgagucggugcG |
| | UUUUAGagcuaGaaauagcaaGUUaAa | AGGUGGUCAUCCGCGAGGUG |
| | AuAaggcuaGUccGUUAucAAcuugga | UCCGGCA*u*c*a |
| | cggac | |
| 19 | c*c*u*UGAUGCCGGACACCUCGg | ccguccaagugGcaccgagucggugcGA |
| | UUUUAGagcuaGaaauagcaaGUUaAa | GGUGGUCAUCCGCGAGGUGU |
| | AuAaggcuaGUccGUUAucAAcuugga | CCGGCA*u*c*a |
| | cgg | |
| 20 | c*c*u*UGAUGCCGGACACCUCGg | gcgcguccaagugGcaccgagucggugcG |
| | UUUUAGagcuaGaaauagcaaGUUaAa | AGGUGGUCAUCCGCGAGGUG |
| | AuAaggcuaGUccGUUAucAAcuugga | UCCGGCA*u*c*a |
| | cgcgc | |
| 21 | c*c*u*UGAUGCCGGACACCUCGg | gcguccaagugGcaccgagucggugcGA |
| | UUUUAGagcuaGaaauagcaaGUUaAa | GGUGGUCAUCCGCGAGGUGU |
| | AuAaggcuaGUccGUUAucAAcuugga | CCGGCA*u*c*a |
| | cgc | |
| 22 | c*c*u*UGAUGCCGGACACCUCGg | guccaagugGcaccgagucggugcGAG |
| | UUUUAGagcuaGaaauagcaaGUUaAa | GUGGUCAUCCGCGAGGUGUC |
| | AuAaggcuaGUccGUUAucAAcuugga | CGGCA*u*c*a |
| | c | |
| 23 | c*c*u*UGAUGCCGGACACCUCGg | g*c*a*CAUGAGGAUCACCCAU |
| | UUUUAGagcuaGaaauagcaaGUUaAa | GUGCGAGGUGGUCAUCCGCG |
| | AuAaggcuaGUccGUUAucAAcuuGaa | AGGUGUCCGGCA*u*c*a |
| | aaagugGcaccgagucggugcu*u*u*u | non-hybridizing control |
| | non-hybridizing control | |

Within this table, “A”, “G”, “C”, and “U” refer to ribonucleotides. “a”, “g”, c”, and “u” refer to 2′-O-methyl modified nucleotides. “*” indicates a phosphorothioate linkage between bases.

Guide 23 in Table 3 above is a non-hybridized control that has a full length standard 100mer single guide (polypeptide 1) and a separate polynucleotide (polynucleotide 2) containing a MS2 stem loop, an editing template, and a PBS, which may be referred to in the literature as a “petRNA”. See, Liu, B. et al. A split prime editor with untethered reverse transcriptase and circular RNA template. Nat Biotechnol 1-6 (2022) doi:10.1038/s41587-022-01255-9. In this non-hybridized control, polynucleotide 1 contains the same spacer sequence as the hybridized guides and is configured to complex with Cas9 (H840A) to create a nick in the LMNA gene. Polynucleotide 2 of the non-hybridized control interacts with the MCP component of the RT protein (encoded by mRNA 2) to allow the RT to write the edit into the nicked strand of the LMNA gene. Guide 23 (the non-hybridized control) has no engineered hybridization, and the spacer and full tracr sequence are located in polynucleotide 1. Guide 23 is engineered to edit according to the mechanism shown in FIG. 1C.

Some of the hybridized guides listed in Table 3 above do not contain an MS2 stem-loop and are compatible with a standard PEmax protein. These guides are engineered to facilitate editing according the mechanism shown in FIG. 1B.

Some of the hybridized guides listed in Table 3 above contain the MS2 stem-loop structure (polynucleotide 2 of guides 12-15 and polynucleotide 1 of guides 16 and 17) and are also engineered to hybridize within the tracr region.

The resulting editing efficiencies are presented below in Table 4.

TABLE 4
Editing Efficiencies

| Hybridized | mRNA | Dose | |
|---|---|---|---|
| guide no. | Configuration | (ng/mL) | Mean ± S.D. |
| 1 | 1 | 315 | 5.2 ± 1.5 |
| 2 | 1 | 315 | 4.4 ± 0.3 |
| 3 | 1 | 315 | 8.8 ± 3.2 |
| 4 | 1 | 315 | 4 ± 0.9 |
| 5 | 1 | 315 | 6.5 ± 2.8 |
| 6 | 1 | 315 | 5.8 ± 3.5 |
| 7 | 1 | 315 | 7.2 ± 1.4 |
| 8 | 1 | 315 | 6.2 ± 2.1 |
| 9 | 1 | 315 | 12.6 ± 2.3 |
| 10 | 1 | 315 | 13.3 ± 3.3 |
| 11 | 1 | 315 | 19.6 ± 2.4 |
| 23 | 1 | 315 | 30.3 ± 3.7 |
| 1 | 2 | 315 | 2.5 ± 3.3 |
| 2 | 2 | 315 | 2.6 ± 1.3 |
| 3 | 2 | 315 | 1.3 ± 0.5 |
| 4 | 2 | 315 | 1.6 ± 1.4 |
| 5 | 2 | 315 | 2.9 ± 1.9 |
| 6 | 2 | 315 | 1.9 ± 1.4 |
| 7 | 2 | 315 | 3.8 ± 2.4 |
| 8 | 2 | 315 | 3.1 ± 2.1 |
| 9 | 2 | 315 | 5.6 ± 0.5 |
| 10 | 2 | 315 | 7.2 ± 5.6 |
| 11 | 2 | 315 | 6.8 ± 5.1 |
| 23 | 2 | 315 | 2.1 ± 1.4 |
| 1 | 1 | 1260 | 8.1 ± 2.8 |
| 2 | 1 | 1260 | 13.6 ± 4.1 |
| 3 | 1 | 1260 | 8 ± 3.4 |
| 4 | 1 | 1260 | 6 ± 4.5 |
| 5 | 1 | 1260 | 7.8 ± 6.5 |
| 6 | 1 | 1260 | 13 ± 0.8 |
| 7 | 1 | 1260 | 11.2 ± 0.3 |
| 8 | 1 | 1260 | 10.3 ± 2.5 |
| 9 | 1 | 1260 | 13.4 ± 2.9 |
| 10 | 1 | 1260 | 19.2 ± 5 |
| 11 | 1 | 1260 | 25.1 ± 1.3 |
| 23 | 1 | 1260 | 38.5 ± 3.8 |
| 1 | 2 | 1260 | 2 ± 1.1 |
| 2 | 2 | 1260 | 4 ± 1.9 |
| 3 | 2 | 1260 | 1.6 ± 1.4 |
| 4 | 2 | 1260 | 4.6 ± 1.7 |
| 5 | 2 | 1260 | 5.4 ± 1.6 |
| 6 | 2 | 1260 | 6.9 ± 1.1 |
| 7 | 2 | 1260 | 7.7 ± 1.1 |
| 8 | 2 | 1260 | 6.7 ± 2.7 |
| 9 | 2 | 1260 | 7.9 ± 2 |
| 10 | 2 | 1260 | 10.7 ± 5.6 |
| 11 | 2 | 1260 | 17.5 ± 6.1 |
| 23 | 2 | 1260 | 4.8 ± 0.6 |
| 12 | 1 | 157.5 | 29.3 ± 3.3 |
| 13 | 1 | 157.5 | 25.3 ± 0.7 |
| 14 | 1 | 157.5 | 36 ± 1.8 |
| 15 | 1 | 157.5 | 32.5 ± 0.3 |
| 16 | 1 | 157.5 | 13.5 ± 1.1 |
| 17 | 1 | 157.5 | 21.8 ± 1.2 |
| 18 | 1 | 157.5 | 19.3 ± 1.6 |
| 19 | 1 | 157.5 | 23.1 ± 4.3 |
| 20 | 1 | 157.5 | 21 ± 0.5 |
| 21 | 1 | 157.5 | 25.3 ± 1.3 |
| 22 | 1 | 157.5 | 13.3 ± 1.8 |
| 23 | 1 | 157.5 | 20.1 ± 2.6 |
| 12 | 1 | 315 | 21.5 ± 1.3 |
| 13 | 1 | 315 | 20.5 ± 2 |
| 14 | 1 | 315 | 34 ± 1.7 |
| 15 | 1 | 315 | 29 ± 9.5 |
| 16 | 1 | 315 | 16.2 ± 5.5 |
| 17 | 1 | 315 | 21.3 ± 6.6 |
| 18 | 1 | 315 | 14.8 ± 4.2 |
| 19 | 1 | 315 | 22 ± 5.1 |
| 20 | 1 | 315 | 16.4 ± 7.7 |
| 21 | 1 | 315 | 22.6 ± 4.6 |
| 22 | 1 | 315 | 16.5 ± 6.8 |
| 23 | 1 | 315 | 15.1 ± 4.6 |

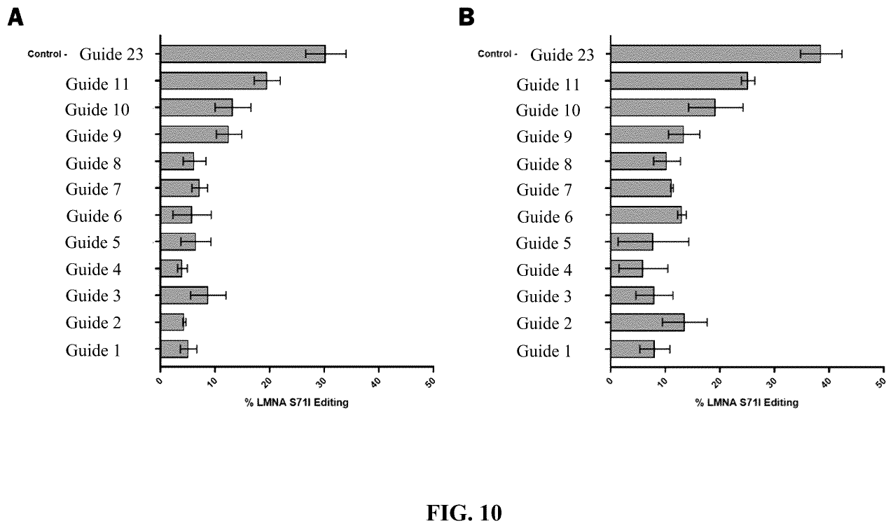

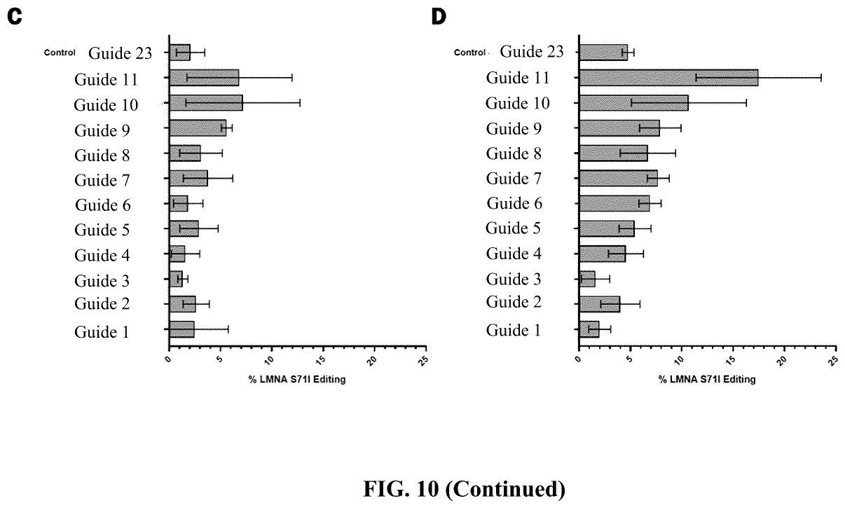

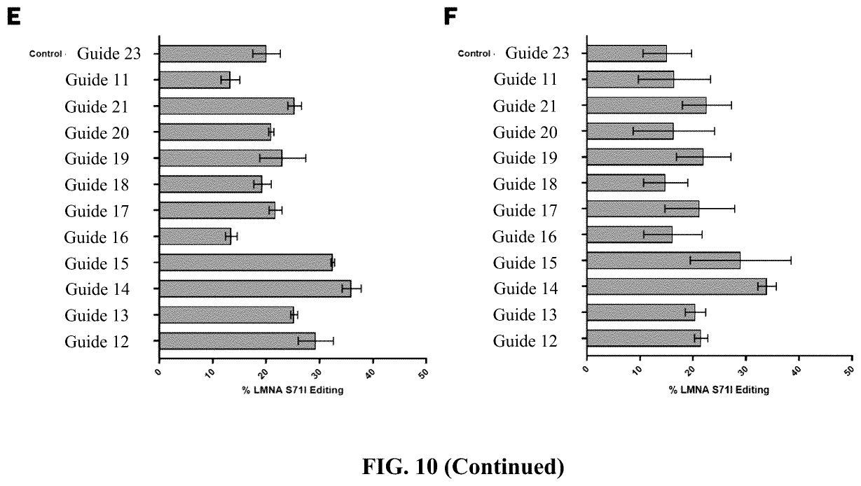

This series of hybridized guides showed a correlation between increased editing efficiency with increased length of the hybridization helix. One possible explanation is that the two halves of the guide were more likely to anneal to each other and reconstitute functional guide under cellular conditions. This trend was observed in both mRNA Configuration 1 and mRNA Configuration 2. The presence of an MS2 loop improves editing efficiency in the context of mRNA Configuration 1, which expresses a protein containing the MCP binding partner for MS2. This suggests that with certain hybridized guide configurations, the addition of an RNA-protein interaction between the guide and the RT domain can improve editing efficiency. Notably, the hybridized guides numbers 7, 8, 9, 10, and 11 showed improved or similar editing efficiency in the absence of MCP (mRNA Configuration 2) over the non-hybridizing control, suggesting that the hybridized guide designs shown here may have an advantage over current split guide precedents in certain contexts, possibly by reconstituting a functional guide via the hybridization rather than exclusively via an MCP-dependent manner. The hybridized guides may also effectively localize the RT template region to the editing site. Editing efficiency is shown in FIG. 10 and Table 4.

What is claimed:

1. A hybridized guide nucleic acid for use with a template-based editor, comprising:
a first polynucleotide comprising a first guide nucleic acid element and a first hybridization sequence; and
a second polynucleotide comprising a second guide nucleic acid element and a second hybridization sequence,
wherein the second guide nucleic acid element is an editing template,
wherein the first and second hybridization sequences are complementary, and
wherein the first and second hybridization sequences are hybridized to one another.

2. The hybridized guide nucleic acid of claim 1, wherein the second polynucleotide further comprises a third hybridization sequence, and wherein the hybridized guide nucleic acid further comprises a third polynucleotide comprising a third guide nucleic acid element and a fourth hybridization sequence,
wherein the third and fourth hybridization sequences are complementary, and
wherein the third and fourth hybridization sequences are hybridized to one another.

3. A hybridized guide nucleic acid for use with a template based-editor, the hybridized guide nucleic acid comprising a plurality of polynucleotides,
wherein each polynucleotide comprises at most 80 nucleotides in length,
wherein each polynucleotide comprises a gRNA element and a hybridization sequence,
wherein at least one of the polynucleotides comprises an editing template;
wherein each hybridization sequence is complementary to at least one other hybridization sequence, and
wherein the plurality of polynucleotides are hybridized to one another to form the hybridized guide nucleic acid, wherein the hybridized guide nucleic acid comprises a plurality of guide nucleic acid elements.

4. The hybridized guide nucleic acid of claim 3, wherein one or more of the polynucleotides comprises more than one guide nucleic acid element.

5. The hybridized guide nucleic acid of claim 3, wherein at least one of the guide nucleic acid elements is a spacer region.

6. The hybridized guide nucleic acid of claim 3, wherein at least one of the guide nucleic acid elements comprises a tracr region or a portion thereof.

7. The hybridized guide nucleic acid of claim 6, wherein at least one of the hybridization sequences is in the tracr region.

8. The hybridized guide nucleic acid of claim 7, wherein the hybridization sequences in the tracr region comprises 4-9 nucleotides.

9. The hybridized guide nucleic acid of claim 7, the hybridization sequence of at least one polynucleotide in the tracr region located internal to a 5′ end and a 3′ end of the polynucleotide.

10. The hybridized guide of claim 9, wherein the at least one polynucleotide comprises a MS2 stem-loop structure.

11. The hybridized guide of claim 10, wherein the hybridization sequence is adjacent the MS2 stem-loop structure.

12. The hybridized guide nucleic acid of any claim 3, wherein one or more of the polynucleotides is chemically modified.

13. The hybridized guide nucleic acid of claim 3, wherein one of the polynucleotides comprises a spacer region, a tracr region up to a first hairpin strand, and the editing template, and another of the polynucleotides comprises a tracr region from a second hairpin to the 3′ end of the tracr region and a second editing template.

14. A method for forming the hybridized guide nucleic acid of claim 3, the method comprising hybridizing each of the polynucleotides to another of the polynucleotides, such that a hybridization sequence of each of the polynucleotides hybridizes a hybridization sequence of another polynucleotide.

15. A template-based gene editor system comprising:
one or more mRNAs encoding a template-based gene editor or components thereof;
a hybridized guide according to claim 3, or components thereof.

16. A pharmaceutical composition comprising the template-based editor system according to claim 15.

17. The pharmaceutical composition of claim 16, further comprising a lipid nanoparticle (LNP).

18. The pharmaceutical composition of claim 17, wherein the lipid nanoparticle comprises the one or more mRNAs encoding a template-based gene editor or components thereof and comprises the hybridized guide or components thereof.

19. A composition comprising:
a plurality of polynucleotides,
wherein each polynucleotide comprises at most 80 nucleotides in length,
wherein each polynucleotide comprises a gRNA element and a hybridization sequence,
wherein at least one of the polynucleotides comprises an editing template;
wherein each hybridization sequence is complementary to at least one other hybridization sequence, and
wherein the plurality of polynucleotides are engineered to hybridize to one another to form a functional guide nucleic acid for use in a template-based guide nucleic acid system.

20. The composition of claim 19, wherein at least one of the guide nucleic acid elements comprises a tracr region or a portion thereof.

21. The composition of claim 20, wherein at least one of the hybridization sequences in in the tracr region.
